(19) United States
(12) Patent Application Publication
(10) Pub. No.: US 2025/0160702 A1
(43) Pub. Date: May 22, 2025

(54) HIGHLY PERMEABLE MEMBRANE FOR BIOSENSOR

(71) Applicant: ABBOTT DIABETES CARE INC., Alameda, CA (US)

(72) Inventors: Tianmei OUYANG, Saratoga, CA (US); Rifat E. OZEL, San Jose, CA (US); Benjamin FELDMAN, Berkeley, CA (US)

(73) Assignee: Abbott Diabetes Care Inc., Alameda, CA (US)

(21) Appl. No.: 18/782,967

(22) Filed: Jul. 24, 2024

Related U.S. Application Data
(60) Provisional application No. 63/587,753, filed on Oct. 4, 2023.

(51) Int. Cl. A61B 5/1486; A61B 5/00; A61B 5/145; A61B 5/1495; A61L 31/04; A61L 31/14

(52) U.S. Cl. CPC A61B 5/14865; A61B 5/002; A61B 5/14532; A61B 5/1495; A61L 31/048; A61L 31/14; A61B 2560/0228; A61L 2400/18

(57) ABSTRACT

The present disclosure provides analyte sensors comprising a first working electrode, a sensing layer disposed upon a surface of the first working electrode, and a highly permeable membrane that overcoats at least a part of the sensing layer and that is permeable to an analyte, wherein the highly permeable membrane comprises a copolymer of poly(N-vinylimidazole) and poly(N-isopropylacrylamide), and wherein the analyte sensor shows a sensitivity of at least 100 nA/mM to the analyte. The present disclosure also provides methods of using such analyte sensors for detecting one or more analytes preset in a biological sample and methods of manufacturing the analyte sensors.

### FIELD

The present disclosure provides analyte sensors comprising a first working electrode, a sensing layer disposed upon a surface of the first working electrode, and a highly permeable membrane that overcoats at least a part of the sensing layer and that is permeable to an analyte, wherein the highly permeable membrane comprises a copolymer of poly(N-vinylimidazole) and poly(N-isopropylacrylamide), and wherein the analyte sensor shows a sensitivity of at least 100 nA/mM to the analyte. The present disclosure also provides methods of using such analyte sensors for detecting one or more analytes present in a biological sample and methods of manufacturing the analyte sensors.

### BACKGROUND

The detection of various analytes within an individual can sometimes be vital for monitoring the condition of their health, as deviations from normal analyte levels can be indicative of a physiological condition. For example, monitoring glucose levels can enable people suffering from diabetes to take appropriate corrective action including administering medicine or consuming a particular food or beverage products to avoid significant physiological harm. Other analytes can be desirable to monitor for other physiological conditions. In some instances, it can be desirable to monitor more than one analyte when monitoring single or multiple physiological conditions, particularly if a person is suffering from comorbid conditions that result in simultaneous dysregulation of two or more analytes in combination with one another.

Analyte monitoring in an individual can take place periodically or continuously over a period of time. Periodic analyte monitoring can take place by withdrawing a sample of bodily fluid, such as blood or urine, at set time intervals and analyzing the same ex vivo. Periodic, ex vivo analyte monitoring can be sufficient to determine the physiological condition of many individuals. However, ex vivo analyte monitoring can be inconvenient or painful in some instances. Moreover, there is no way to recover lost data if an analyte measurement is not obtained at an appropriate time.

Continuous analyte monitoring can be conducted using one or more sensors that remain at least partially implanted within a tissue of an individual, such as dermally, subcutaneously, or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well. Since implanted analyte sensors often remain within a tissue of an individual for an extended period of time, it can be highly desirable for such analyte sensors to be made from stable materials exhibiting a high degree of biocompatibility.

Sensors can include a membrane disposed over at least the implanted portion of the sensor. In one aspect, the membrane can improve biocompatibility of the sensor in vivo. In another aspect, the membrane can be permeable or semi-permeable to an analyte of interest but limit the overall flux of the analyte to the active sensing portion of the sensor. One difficulty associated with incorporating a membrane upon an analyte sensor is that the analyte flux across the membrane can vary considerably as a function of temperature. While a calibration factor or equation can be employed to account for analyte flux variability as a function of temperature, doing so can add considerable complexity to the use of the sensor, especially if the flux is non-linear with respect to temperature. Moreover, thermistors used in applying a calibration equation can be complicated to operate and their size can thwart efforts to minimize the size of the sensors. As another difficulty, the calibration temperature measurement location can have a different temperature than that of the membrane covering an active portion of the sensor. Other components of the sensor can likewise exhibit performance variability due to temperature (e.g., the enzymatic reaction rate in the case of an enzyme-based sensor), which can make isolation and application of a calibration factor or equation for the membrane rather difficult. Accordingly, there is a need for membranes that allow analytes to readily permeate through the membrane and also exhibit little variation due to temperature.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The present disclosure relates to an analyte sensor comprising a first working electrode, a first sensing layer disposed upon a surface of the first working electrode, and a membrane covering at least a part of the first sensing layer. In some embodiments, the membrane overcoats at least a part of the first working electrode.

The membrane comprises a copolymer of poly(N-vinylimidazole) and poly(N-isopropylacrylamide). In some embodiments, the membrane comprises a block copolymer of poly(N-vinylimidazole) and poly(N-isopropylacrylamide). In some embodiments, the membrane comprises a copolymer comprising a poly(N-vinylimidazole) block having a number average molecular weight from about 2800 to about 4800 (e.g., from about 3300 to about 4200, e.g., about 3800) and a poly(N-isopropylacrylamide) block having a number average molecular weight from about 5600 to about 8000 (e.g., from about 6200 to about 7400, e.g., about 6800).

In some embodiments, the membrane comprises a crosslinking agent. In some embodiments, the crosslinking agent is a polyethylene glycol diglycidylether (PEGDGE). In some embodiments, the ratio (w/v) of the crosslinking agent to the copolymer of poly(N-vinylimidazole) and poly(N-isopropylacrylamide) is from about 1:100 to about 1:1, e.g., from about 1:40 to about 1:20. The membrane is typically permeable, e.g., is permeable to the analyte. In some embodiments, the membrane is highly permeable (e.g., is highly permeable to the analyte) as described in more detail herein.

In some embodiments, the analyte sensor is a sensor for an analyte selected from glucose, glutamate, a ketone, an alcohol, lactate, and combinations thereof, e.g., for glucose or glutamate. In some embodiments the analyte sensor is configured to sense the analyte (i.e. the sensor is responsive to the analyte).

The membrane is typically stable at temperatures from about 22° C. to about 42° C. (e.g., in phosphate buffered saline). In some embodiments, the membrane comprises a copolymer as described herein which has a lower critical solution temperature (LCST) of from about 22° C. to about 42° C.

In some embodiments, the sensor has a sensitivity of at least 100 nA/mM. This is described in more detail herein. Furthermore, the sensor is typically stable to delamination. In some embodiments, the analyte sensor exhibits less than 20% delamination over a period of 12 days, e.g., less than 5% delamination over a period of 15 days.

In some embodiments, the analyte sensor further comprises a second sensing layer disposed upon a surface of the first working electrode, the second sensing layer being responsive to a second analyte differing from the first analyte. In some embodiments the second sensing layer comprises at least one enzyme responsive to the second analyte. In some embodiments, the analyte sensor further comprises a second working electrode; and a second sensing layer disposed upon a surface of the second working electrode, the second sensing layer being responsive to a second analyte differing from the first analyte. In some embodiments, the second sensing layer comprises at least one enzyme responsive to the second analyte.

Accordingly, the present disclosure provides an analyte sensor comprising:
(i) a first working electrode;
(ii) a first sensing layer disposed upon a surface of the first working electrode; and
(iii) a membrane overcoating at least a part of the first sensing layer and that is permeable to a first analyte, the membrane comprising a copolymer of poly(N-vinylimidazole) and poly(N-isopropylacrylamide).

In some embodiments, the membrane is a highly permeable membrane.

In some embodiments, the analyte sensor shows a sensitivity of at least 100 nA/mM to the first analyte.

The present disclosure provides an analyte sensor comprising:
(i) first working electrode;
(ii) a first sensing layer disposed upon a surface of the first working electrode; and
(iii) a highly permeable membrane that overcoats at least a part of the first sensing layer and that is permeable to a first analyte, wherein the highly permeable membrane comprises a copolymer of poly(N-vinylimidazole) and poly(N-isopropylacrylamide);

wherein the analyte sensor shows a sensitivity of at least 100 nA/mM to the first analyte.

In some embodiments, the analyte sensor comprises a highly permeable membrane that exhibits a lower critical solution temperature over a range from about 22° C. to about 42° C. in phosphate buffered saline.

In some embodiments, the analyte sensor comprises a highly permeable membrane comprising a copolymer with a poly(N-vinylimidazole) block having a number average molecular weight from about 2800 to about 4800 and a poly(N-isopropylacrylamide) block having a number average molecular weight from about 5600 to about 8000.

In some embodiments, the analyte sensor comprises a highly permeable membrane comprising a copolymer with a poly(N-vinylimidazole) block having a number average molecular weight from about 3300 to about 4200 and a poly(N-isopropylacrylamide) block having a number average molecular weight from about 6200 to about 7400.

In some embodiments, the analyte sensor comprises a highly permeable membrane comprising a copolymer with a poly(N-vinylimidazole) block having a number average molecular weight of about 3800 and a poly(N-isopropylacrylamide) block having a number average molecular weight of about 6800.

In some embodiments, the analyte sensor comprising a highly permeable membrane further comprises a crosslinking agent.

In some embodiments, the analyte sensor comprising a highly permeable membrane further comprises a crosslinking agent, wherein the crosslinking agent is a polyethylene glycol diglycidylether (PEGDGE).

In some embodiments, the ratio (w/v) of the crosslinking agent to the copolymer of poly(N-vinylimidazole) and poly(N-isopropylacrylamide) is from about 1:100 to about 1:1.

In some embodiments, the ratio (w/v) of the crosslinking agent to the copolymer of poly(N-vinylimidazole) and poly(N-isopropylacrylamide) is from about 1:40 to about 1:20.

In some embodiments, the analyte sensor generates a signal that is substantially temperature independent over a range of temperatures.

In some embodiments, the analyte sensor generates a signal that is substantially temperature independent over a range of temperatures, wherein the range of temperatures is from about 25° C. to about 45° C.

In some embodiments, the analyte sensor generates a signal that varies by no more than 5% over the temperature range at a constant analyte concentration.

In some embodiments, the analyte sensor shows a sensitivity of at least 150 nA/nM to the first analyte.

In some embodiments, the analyte sensor shows a sensitivity to an analyte that is greater than the sensitivity to the analyte shown by an otherwise identical sensor lacking a highly permeable membrane comprising a copolymer of poly(N-vinylimidazole) and poly(N-isopropylacrylamide).

In some embodiments, the analyte sensor shows a sensitivity to an analyte that is at least 25% greater than the sensitivity to the analyte shown by an otherwise identical sensor lacking a highly permeable membrane comprising a copolymer of poly(N-vinylimidazole) and poly(N-isopropylacrylamide).

In some embodiments, the analyte sensor generates a signal that varies by no more than 10% over the temperature range at a constant analyte concentration for at least 10 days.

In some embodiments, the analyte sensor generates a signal that varies by no more than 10% over the temperature range of from about 25° C. to about 45° C. at a constant analyte concentration for at least 15 days.

In some embodiments, the analyte sensor detects a first analyte selected from the group consisting of glucose, glutamate, a ketone, an alcohol, lactate, and combinations thereof.

In some embodiments, the first analyte is glucose.

In some embodiments, the first analyte is glutamate.

In some embodiments, the analyte sensor comprises a first working electrode, wherein the first working electrode comprises carbon.

In some embodiments, the analyte sensor comprises a first sensing layer, wherein the first sensing layer further comprises a redox mediator.

In some embodiments, the analyte sensor further comprising a reference electrode, a counter electrode, or both a reference electrode and a counter electrode.

In some embodiments, the analyte sensor exhibits less than 20% delamination over a period of 12 days.

In some embodiments, the analyte sensor exhibits less than 5% delamination over a period of 15 days.

In some embodiments, the analyte sensor further comprises a second sensing layer disposed upon a surface of the first working electrode, the second sensing layer being responsive to a second analyte differing from the first analyte, wherein the first sensing layer comprises at least one enzyme responsive to the first analyte and the second sensing layer comprises at least one enzyme responsive to the second analyte.

The present disclosure provides an analyte sensor comprising:
(i) first working electrode;
(ii) a first sensing layer disposed upon a surface of the first working electrode;
(iii) a highly permeable membrane that overcoats at least a part of the first sensing layer and that is permeable to a first analyte; wherein the analyte sensor shows a sensitivity of at least 100 nA/mM to the first analyte;
(iv) a second working electrode; and
(v) a second sensing layer disposed upon a surface of the second working electrode, the second sensing layer being responsive to a second analyte differing from the first analyte; wherein the first sensing layer comprising at least one enzyme responsive to the first analyte and the second sensing layer comprises at least one enzyme responsive to the second analyte.

In some embodiments, a highly permeable membrane overcoats at least portion of the second sensing layer and is permeable to the second analyte; wherein the analyte sensor shows a sensitivity of at least 100 nA/mM to the second analyte.

The present disclosure provides a method for monitoring a level of an analyte comprising:
(i) applying a potential to a first working electrode of an analyte sensor, wherein the analyte sensor comprises:
   (a) a first working electrode;
   (b) a first sensing layer disposed upon a surface of the first working electrode; and
   (c) a highly permeable membrane that overcoats at least a part of the first sensing layer and that is permeable to a first analyte; wherein the highly permeable membrane comprises a copolymer of poly(N-vinylimidazole) and poly(N-isopropylacrylamide); and wherein the analyte sensor shows a sensitivity of at least 100 nA/mM to the first analyte;
(ii) obtaining a first signal at or above an oxidation-reduction potential of the first sensing layer, the first signal being proportional to a concentration of a first analyte in a fluid contacting the first sensing layer; and
(iii) correlating the first signal to the concentration of the first analyte in the fluid.

In some embodiments, the method comprises an analyte sensor comprising a copolymer of poly(N-vinylimidazole) and poly(N-isopropylacrylamide) that exhibits a lower critical solution temperature over a range from about 22° C. to about 42° C. in phosphate buffered saline.

### BRIEF DESCRIPTION OF THE DRAWINGS

The following figures are included to illustrate some aspects of the present disclosure and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that can incorporate an analyte sensor of the present disclosure.

FIGS. 2A-2C show cross-sectional diagrams of analyte sensors including a single sensing layer.

FIGS. 3A-3C show cross-sectional diagrams of analyte sensors including two sensing layers.

FIG. 4 shows a cross-sectional diagram of an analyte sensor including two sensing layers.

FIGS. 5A-5C show perspective views of analyte sensors including two sensing layers upon separate working electrodes.

FIG. 6A is a line graph showing sensor current (nA) versus time (hours) of exemplary glucose sensors of the present disclosure comprising the highly permeable membrane (HPA) of Example 4, the highly permeable membrane (HPB) of Example 5, and the control membrane (Control) of Example 2.

FIG. 6B is a line graph showing sensor current (nA) versus time (hours) of exemplary glucose sensors of the present disclosure comprising the highly permeable membrane (HPA) of Example 4, the highly permeable membrane (HPB) of Example 5, and the control membrane (Control) of Example 2. The sensor current (nA) of FIG. 6A is expanded to show a lower current range to allow the current for the control membrane to be detected.

FIG. 7A is a line graph showing the sensor current (nA) versus concentration of exemplary glucose sensors of the present disclosure comprising the highly permeable membrane (HPA) of Example 4, the highly permeable membrane (HPB) of Example 5, and the control membrane (Control) of Example 2. The left-hand y-axis provides the current range for the two highly permeable membranes (HPA and HPB) and the right-hand y-axis provides the current range for the control membrane (Control).

FIG. 7B is a bar graph showing the sensitivity (nA/mM) of exemplary glucose sensors of the present disclosure comprising the highly permeable membrane (HPA) of Example 4, the highly permeable membrane (HPB) of Example 5, and the control membrane (Control) of Example 2.

FIG. 8A is a line graph showing the current (nA) versus time (hours) of an exemplary control glucose sensor. The graph shows the effect of temperature at a range from 22° C. to 42° C. on the exemplary control glucose sensor.

FIG. 8B is a line graph showing the current (nA) versus time (hours) of exemplary glucose sensors of the present disclosure comprising the highly permeable membrane (HPA) of Example 4 and the highly permeable membrane (HPB) of Example 5. The graph shows that at a range from 22° C. to 42° C. the exemplary glucose sensors comprising highly permeable membranes are relatively insensitive to temperature.

FIG. 9 shows a diagram of a glutamate enzyme system that can be used for detecting glutamate according to the present disclosure.

FIG. 10A is a line graph showing the current (nA) versus time (hours) of exemplary glutamate sensors of the present disclosure comprising the highly permeable membrane (HPC) of Example 9 and the control membrane (10Q5) of Example 7.

FIG. 10B is a line graph of FIG. 10A showing the current (nA) versus time (hours) of exemplary glutamate sensors of the present disclosure comprising the highly permeable membrane (HPC) of Example 8 and the control membrane (10Q5) of Example 9. The sensor current (nA) of FIG. 10A is expanded to show a lower current range to allow the current for the control membrane to be detected.

FIG. 11A is a line graph showing the sensor current (nA) versus concentration of glutamate of exemplary glutamate sensors comprising the highly permeable membrane (HPC) of Example 9 and the control membrane (10Q5) of Example 7.

FIG. 11B is a bar graph showing the sensitivity (nA/mM) of exemplary glutamate sensors of the present disclosure comprising the highly permeable membrane (HPC) of Example 9 and the control membrane (10Q5) of Example 7.

FIG. 12A is a line graph showing the current (nA) of an exemplary glutamate sensor of the present disclosure comprising the highly permeable membrane (HPC) of Example 9 over 15 days with measurements shown at Day 1 and at Day 15. As shown in the graph, the current only decreased by 5% after 15 days of use.

FIG. 12B is a bar graph showing the sensitivity (nA/mM) of an exemplary glutamate sensor of the present disclosure comprising the highly permeable membrane (HPC) of Example 8 at Day 1 and at Day 15.

FIG. 13 is a line graph showing the current (nA) versus concentration (mM) of the glucose sensor of Example 12 coated with a membrane comprising poly(4-vinylpyridine-co-N-isopropylacrylamide). The slope of the dotted line indicates that the sensitivity of the glucose sensor coated with the membrane comprising poly(4-vinylpyridine-co-N-isopropylacrylamide) was about 1.5 nA/mM.

FIG. 14 is a line graph showing the current (nA) versus concentration (mM) of the glucose sensor of Example 12 coated with a membrane comprising poly(4-vinylimidazole-co-N-isopropylacrylamide). The slope of the dotted line indicates that the sensitivity of the glucose sensor coated with the membrane comprising poly(4-vinylimidazole-co-N-isopropylacrylamide) was about 270 nA/mM.

FIG. 15 is a bar graph comparing the sensitivity measured for the glucose sensor of Example 12 coated with a membrane comprising poly(4-vinylpyridine-co-N-isopropylacrylamide) to the sensitivity measured for the glucose sensor of Example 12 coated with a membrane comprising poly(4-vinylimidazole-co-N-isopropylacrylamide).

### DETAILED DESCRIPTION

The present disclosure is directed to analyte sensors comprising a membrane that covers (e.g., overcoats) at least a part of the sensing layer and that is permeable to an analyte; wherein the membrane comprises a copolymer of poly(N-vinylimidazole) and poly(N-isopropylacrylamide). The present disclosure is also directed to analyte sensors comprising a membrane (e.g., a highly permeable membrane) that overcoats at least a part of the sensing layer and that is permeable to an analyte; wherein the membrane (e.g., highly permeable membrane) comprises a copolymer of poly(N-vinylimidazole) and poly(N-isopropylacrylamide); and wherein the analyte sensor shows a sensitivity of at least 100 nA/mM to the analyte. The analyte sensors of the present disclosure also exhibit limited analyte permeability variation as a function of temperature. The analyte sensors of the present disclosure further exhibit low delamination.

One difficulty associated with analyte sensors is that some analytes are present in vivo in very low concentrations and thus, are difficult to detect. For example, glutamate is present in plasma at concentrations of about 150 μm. Due to the low concentrations of glutamate present in vivo, in can be very difficult for many analyte sensors to detect the presence of this analyte.

Another difficulty associated with many membrane materials is that their analyte permeability can vary to a clinically significant degree as a function of temperature. Analyte permeability variation as a function of temperature can lead to problematic sensor calibration, especially if the permeability variation is non-linear with respect to temperature. While some membrane materials are known to exhibit limited analyte permeability variation as a function of temperature, they can suffer from biocompatibility issues. Further, some membrane materials can be difficult to purify following synthesis.

Using a membrane material that shows limited analyte permeability variation as a function of temperature can allow the analyte sensor to operate at a higher temperature. By increasing the temperature, the permeability of the membrane should also increase. As the temperature increases, more water molecules form hydrogen-bonds with the polar groups of the polymer network and the membrane expands. This expansion of the membrane results in more analyte diffusing through the membrane and thus, a higher sensitivity of the analyte sensor for an analyte.

The present disclosure provides polymeric membrane compositions that exhibit a desirable combination of limited analyte permeability variation as a function of temperature and high analyte permeability-allowing for detection of analytes that are present in low concentrations.

Another difficulty associated with analyte sensors is that the polymeric membranes used in some sensors are prone to delamination (i.e. loss of coating adhesion). The polymeric membrane compositions of the present disclosure are further advantageous in that they typically demonstrate low delamination.

The present disclosure further provides methods of detecting an analyte using the disclosed sensors and methods of manufacturing the disclosed analyte sensors.

### I. DEFINITIONS

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means±10% of the specified value, unless otherwise indicated.

The term “at least” prior to a number or series of numbers is understood to include the number associated with the term “at least,” and all subsequent numbers or integers that could logically be included, as clear from context. When “at least” is present before a series of numbers or a range, it is understood that “at least” can modify each of the numbers in the series or range. For example, “at least 3” means at least 3, at least 4, at least 5, etc. When “at least” is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the terms “measure,” “measuring,” and “measured” can encompass the meaning of a respective one or more of the terms “determine,” “determining,” “determined,” “calculate,” “calculating,” and “calculated.”

As used herein, an “analyte” is a substance that is subject to be measured or detected. The analyte can be from, for example, a biofluid and can be tested in vivo, ex vivo, or in vitro. Glucose, glutamate, ketones, alcohols, lactate, creatinine, and combinations thereof are exemplary analytes in the present disclosure.

As used herein, a “sensor” is a device configured to detect the presence and/or measure the level of an analyte in a sample via electrochemical oxidation and reduction reactions on the sensor. These reactions are transduced to an electrical signal that can be indicative of presence and be correlated to an amount, concentration, or level of an analyte in the sample.

As used herein, a “working electrode” is an electrode at which the analyte (or additional compound(s) whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated.

As used herein, a “reference electrode” includes both a) reference electrodes and b) reference electrodes that also function as counter electrodes (i.e., counter/reference electrodes), unless otherwise indicated.

As used herein, “electrolysis” is the electrooxidation or electroreduction of a compound either directly at an electrode or via one or more electron transfer agents.

As used herein, an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

A “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, a “sensing layer” is a component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some embodiments of the present disclosure, a sensor includes a sensing layer that is non-leachably disposed in proximity to or on the working electrode. As used herein, the terms “sensing layer” and “sensing area” can be used interchangeably.

As used herein, a “sensing element” is an application or region of an analyte-specific reactant disposed with the sensing layer. As such, a sensing element is capable of interacting with the analyte. A sensing layer can have more than one sensing element making up the analyte detection area disposed on the working electrode. In some embodiments, the sensing element includes an analyte-specific reactant and an electron transfer agent (e.g., electron transfer agent). In some embodiments, the sensing element includes an analyte-specific reactant, a redox mediator, and a crosslinker.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

A “membrane solution” is a solution that contains the components for crosslinking and forming the membrane, including, e.g., polymer (e.g., a modified polymer containing heterocyclic nitrogen groups), a crosslinking agent, and a solvent (e.g., a buffer or an alcohol-buffer mixed solvent).

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears.

The term “patient” refers to a living animal, and thus encompasses a living mammal and a living human, for example. The term “user” can be used herein as a term that encompasses the term “patient.”

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

The phrase “enzyme composition” refers to a composition that includes one or more enzymes for detecting and/or measuring an analyte. In some non-limiting embodiments, the enzyme compositions can include one or more enzymes, polymers, redox mediators, and/or crosslinkers.

As used herein, the phrase “multi-component membrane” refers to a membrane comprising two or more types of membrane polymers.

As used herein, the phrase “multilayered membrane” refers to a membrane system comprising two of more layers of membrane polymer. The two or more layers of membrane polymer can comprise multiple layers of the same membrane polymer as long as there is at least one different membrane polymer layer between the two membrane polymer layers comprising the same membrane polymer.

As used herein, the term “delamination” refers to a loss of coating adhesion to a surface or between coating layers. In some embodiments, delamination refers to the loss of coating adhesion between a sensing layer and a substrate. In some embodiments, delamination refers to the loss of coating adhesion between a sensing layer and a working electrode. In some embodiments, delamination refers to the loss of coating adhesion between a membrane (e.g., highly permeable membrane) and a sensing layer.

As used herein, the term “permeable” in relation to a membrane refers to the extent to which the membrane permits transport (e.g., diffusion) of substrate (permeate) through the membrane.

As used herein, the term “NAD(P)” refers to the nicotinamide adenine dinucleotides NAD⁺ (and its reduced form NADH) and/or NADP⁺ (and its reduced form NADPH). NAD⁺ and NADP⁺ are electron acceptors and NADH and NADPH are electron donors.

As used herein, the phrase “NAD(P)-dependent enzyme” refers to an enzyme that uses NAD⁺ (and its reduced form NADH) and/or NADP⁺ (and its reduced form NADPH) as a cofactor in a redox reaction.

As used herein the term “FAD (P)” refers to the flavin adenine dinucleotides FAD⁺ (and its reduced form FADH) and/or FADP⁺ (and its reduced form FADPH). FAD⁺ and FADP⁺ are electron acceptors and FADH and FADPH are electron donors.

As used herein, the phrase “FAD (P)-dependent enzyme” refers to an enzyme that uses FAD⁺ (and its reduced form FADH) and/or FADP⁺ (and its reduced form FADPH) as a cofactor in a redox reaction.

### II. ANALYTE SENSORS

### Sensors, Compositions, and Methods of the Disclosure

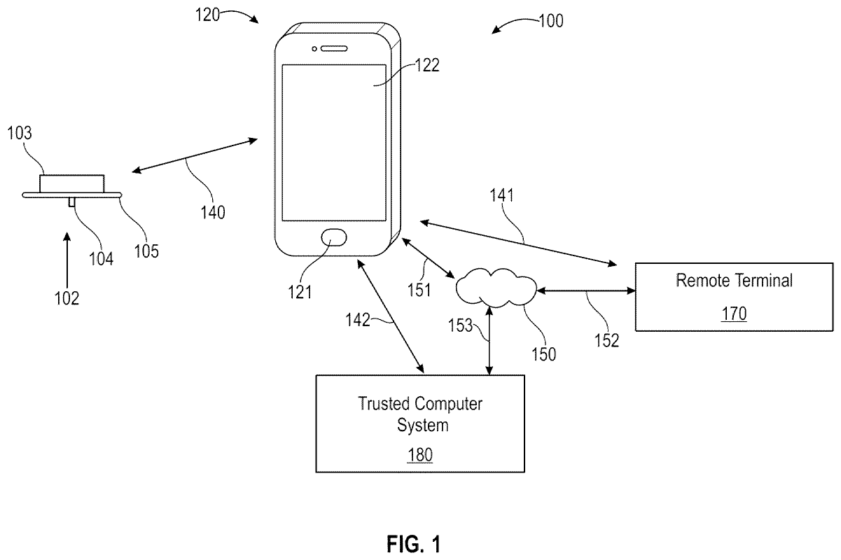

Before describing the analyte sensors of the present disclosure and their components in further detail, a brief overview of suitable in vivo analyte sensor configurations and sensor systems employing the analyte sensors will be provided so that the embodiments of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to some embodiments. Reader device 120 can be a multi-purpose smartphone or a dedicated electronic reader instrument. While only one reader device 120 is shown, multiple reader devices 120 can be present in some instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to some embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to some embodiments, by individuals other than a primary user who have an interest in the user's analyte levels. Reader device 120 can include display 122 and optional input component 121. Display 122 can include a touch-screen interface, according to some embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

Sensor 104 is adapted to be at least partially inserted into a tissue of interest, such as within the dermal or subcutaneous layer of the skin, wherein sensor 104 can comprise a proximal portion and a distal portion. In some embodiments, for example, the distal portion of the sensor can be configured for in vivo placement (e.g., for transcutaneous positioning through the skin of a subject). In some embodiments, an introducer (e.g., a needle or a sharp) can create an insertion path through the subject's skin during the transcutaneous positioning of the distal portion of the sensor). In some embodiments, the sensor can comprise a member capable of penetrating the skin of a subject. In some embodiments, the member can comprises an insertable tip, tail, probe, or needle capable of penetrating the skin of a subject. In some embodiments, the distal portion of sensor 104 can comprise an implantable portion of sufficient length for insertion to a desired depth in a given tissue. The implantable portion (e.g., sensor tail) can include at least one working electrode. In some configurations, the implantable portion (e.g., sensor tail) can include a sensing layer for detecting an analyte. A counter electrode can be present in combination with the at least one working electrode. Particular electrode configurations upon the implantable portion (e.g., sensor tail) are described in more detail below. In another embodiment, the proximal portion of the sensor can be configured to remain above the skin (ex vivo) and can be configured to be electrically coupled with the circuitry disposed in the sensor housing 103 of sensor control device 102.

The sensing layer can be configured for detecting a particular analyte. For example, but not by way of limitation, the disclosed analyte sensors include at least one sensing layer configured to detect an analyte (e.g., glucose, ketone). In some embodiments, a sensor of the present disclosure includes two sensing layers, where each sensing layer is configured to detect a different analyte. Alternatively, the two sensing layers can be configured to detect the same analyte. In some embodiments, a first sensing layer can be configured to detect an analyte (e.g., glucose) and a second sensing layer can be configured to detect the first (i.e., same) analyte or a second analyte different from the first analyte (e.g., ketone, creatinine).

In some embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In some embodiments, analyte sensors of the present disclosure can be adapted for assaying dermal fluid or interstitial fluid to determine a concentration of one or more analytes in vivo. In some embodiments, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a some frequency as data is obtained or after some time period has passed, with the data being stored in a memory until transmittal (e.g., every minute, five minutes, or other predetermined time period). In some embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In some embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In some illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In some embodiments, suitable needles can be solid or hollow, beveled or non-beveled, and/or circular or non-circular in cross-section. In some embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In some embodiments, a tip of the needle (while present) can be angled over the terminus of sensor 104, such that the needle penetrates a tissue first and opens an access pathway for sensor 104. In some embodiments, sensor 104 can reside within a lumen or groove of the needle, with the needle similarly opening an access pathway for sensor 104. In either case, the needle is subsequently withdrawn after facilitating sensor insertion.

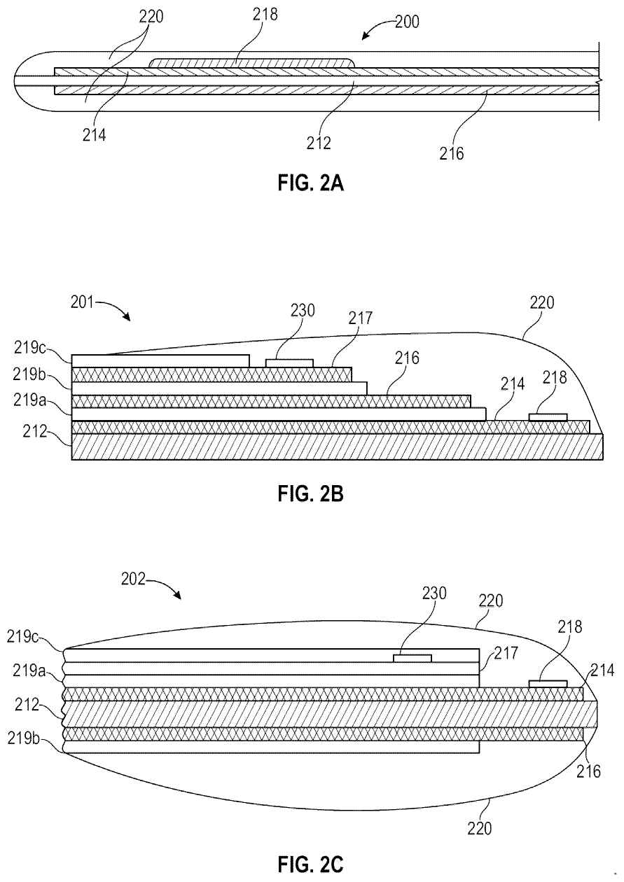

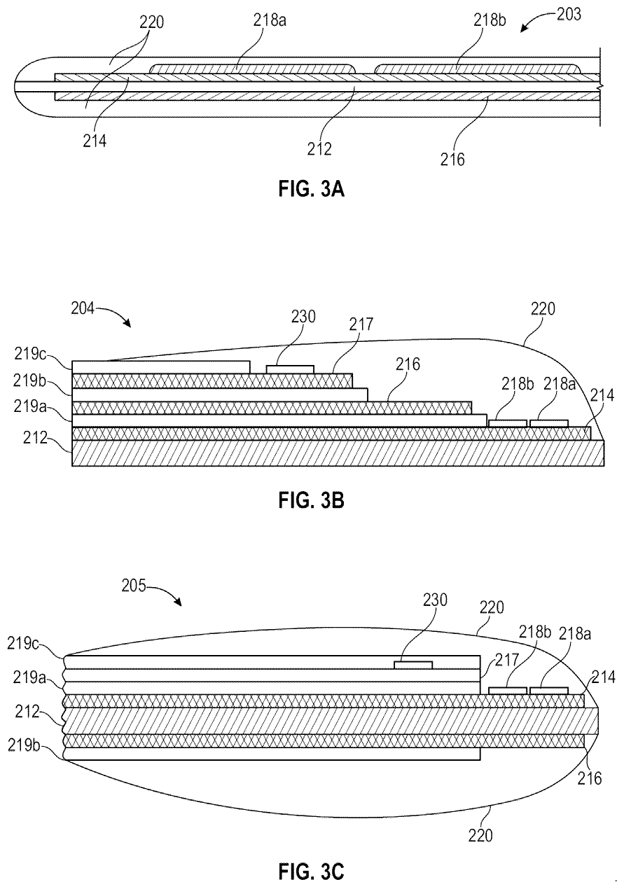

Sensor configurations featuring a single sensing layer that is configured for the detection of a corresponding single analyte can employ two-electrode or three-electrode detection motifs, as described further herein in reference to FIGS. 2A-2C. Sensor configurations featuring two different sensing layers for detection of separate analytes, either upon separate working electrodes or upon the same working electrode, are described separately thereafter in reference to FIGS. 3A-5C. Sensor configurations having multiple working electrodes can be particularly advantageous for incorporating two different sensing layers within the same implantable portion (e.g., sensor tail), since the signal contribution from each sensing layer can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the implantable portion (e.g., sensor tail). Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Sensing layer 218 is disposed as at least one layer upon at least a portion of working electrode 214. Sensing layer 218 can include multiple spots or a single spot configured for detection of an analyte, as discussed further herein.

Referring still to FIG. 2A, membrane 220 overcoats at least sensing layer 218. In some embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to sensing layer 218 (i.e., membrane 220 is a mass transport limiting membrane having some permeability for the analyte of interest). In some embodiments, and further described below, membrane 220 is not crosslinked. Analyte sensor 200 can be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in some embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, sensing layer 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat sensing layer 218, as well as other sensor components, in analyte sensors 201 and 202, thereby serving as a mass transport limiting membrane. In some embodiments, the additional electrode 217 can be overcoated with membrane 220. Although FIGS. 2B and 2C have depicted electrodes 214, 216, and 217 as being overcoated with membrane 220, it is to be recognized that in some embodiments only working electrode 214 is overcoated. Moreover, the thickness of membrane 220 at each of electrodes 214, 216, and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the embodiments disclosed herein, with alternative electrode and/or layer configurations remaining within the scope of the present disclosure.

FIG. 3A shows an illustrative configuration for sensor 203 having a single working electrode with two different sensing layers disposed thereon. FIG. 3A is similar to FIG. 2A, except for the presence of two sensing layers upon working electrode 214: first sensing layer 218a and second sensing layer 218b, which are responsive to different analytes and are laterally spaced apart from one another upon the surface of working electrode 214. Sensing layers 218a and 218b can include multiple spots or a single spot configured for detection of each analyte. The composition of membrane 220 can vary or be compositionally the same at sensing layers 218a and 218b. First sensing layer 218a and second sensing layer 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first sensing layer 218a and second sensing layer 218b disposed thereon. FIGS. 3B and 3C are otherwise similar to FIGS. 2B and 2C and can be better understood by reference thereto. As with FIG. 3A, the composition of membrane 220 can vary or be compositionally the same at sensing layers 218a and 218b.

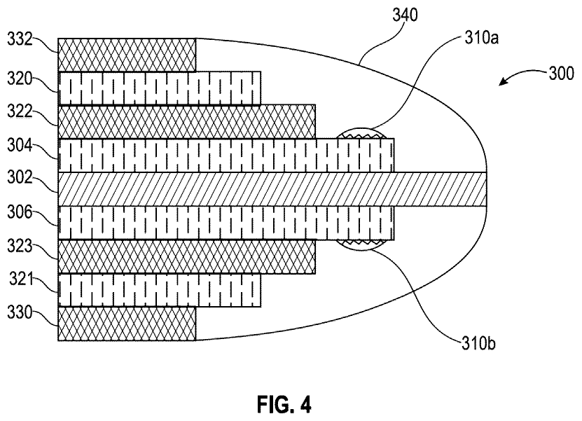

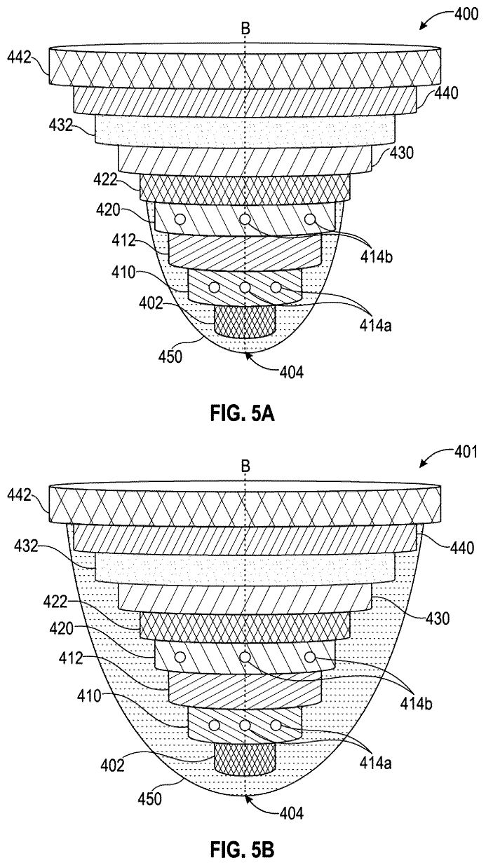

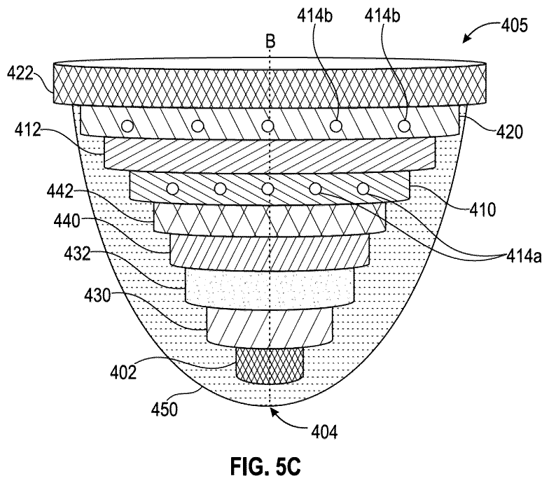

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First sensing layer 310a is disposed upon the surface of working electrode 304, and second sensing layer 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least sensing layers 310a and 310b, according to various embodiments, with other components of analyte sensor 300 or the entirety of analyte sensor 300 optionally being overcoated with membrane 340.

Like analyte sensors 200, 201, and 202, analyte sensor 300 can be operable for assaying an analyte by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described herein below. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first sensing layers 414a and second sensing layers 414b, which are responsive to different analytes or the same analyte, are disposed upon the exposed surfaces of working electrodes 410 and 420, respectively, thereby allowing contact with a fluid to take place for sensing. Although sensing layers 414a and 414b have been depicted as three discrete spots in FIG. 5A, it is to be appreciated that fewer or greater than three spots, including a continuous layer of sensing layer, can be present in alternative sensor configurations.

In FIG. 5A, sensor 400 is partially coated with membrane 450 upon working electrodes 410 and 420 and sensing layers 414a and 414b disposed thereon. FIG. 5B shows an alternative sensor configuration in which the substantial entirety of sensor 401 is overcoated with membrane 450. Membrane 450 can be the same or vary compositionally at sensing layers 414a and 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of sensing layers 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

### III. GENERAL STRUCTURE OF THE ANALYTE SENSOR SYSTEM

In some embodiments, the present disclosure is directed to an analyte sensor system comprising:
(i) a first working electrode;
(ii) a first sensing area disposed upon a surface of the first working electrode; and
(iii) a membrane that covers (e.g., overcoats) at least a part of the first sensing area and that is permeable to a first analyte; wherein the membrane comprises a copolymer of poly(N-vinylimidazole) and poly(N-isopropylacrylamide).

In some embodiments, the present disclosure is directed to a analyte sensor comprising:
(i) first working electrode;
(ii) a first sensing layer disposed upon a surface of the first working electrode; and
(iii) a highly permeable membrane that overcoats at least a part of the first sensing layer and that is permeable to a first analyte; wherein the highly permeable membrane comprises a copolymer of poly(N-vinylimidazole) and poly(N-isopropylacrylamide);
wherein the analyte sensor shows a sensitivity of at least 100 nA/mM to the first analyte.

In some embodiments, the present disclosure is directed to an analyte sensor comprising:
(i) a first working electrode;
(ii) a first sensing area disposed upon a surface of the first working electrode;
(iii) a membrane that covers (e.g., overcoats) at least a part of the first sensing area and that is permeable to a first analyte; wherein the membrane comprises a copolymer of poly(N-vinylimidazole) and poly(N-isopropylacrylamide);

(iv) a second working electrode; and

(v) a second sensing area disposed upon a surface of the second working electrode, the second sensing area being responsive to a second analyte differing from the first analyte; wherein the first sensing area comprises at least one enzyme responsive to the first analyte and the second sensing area comprises at least one enzyme responsive to the second analyte.

In some embodiments, the present disclosure is directed to a analyte sensor comprising:
(i) first working electrode;
(ii) a first sensing layer disposed upon a surface of the first working electrode;
(iii) a highly permeable membrane that overcoats at least a part of the first sensing layer and that is permeable to a first analyte; wherein the highly permeable membrane comprises a copolymer of poly(N-vinylimidazole) and poly(N-isopropylacrylamide); wherein the analyte sensor shows a sensitivity of at least 100 nA/mM to the first analyte;
(iv) a second working electrode; and
(v) a second sensing layer disposed upon a surface of the second working electrode, the second sensing layer being responsive to a second analyte differing from the first analyte; wherein the first sensing layer comprises at least one enzyme responsive to the first analyte and the second sensing layer comprises at least one enzyme responsive to the second analyte.

Embodiments of the present disclosure relate to systems for improving the performance of one or more components of a sensor by inclusion of a membrane system configured to have an analyte permeability that is substantially temperature insensitive, i.e., that does not substantially vary with changes in temperature, and that allows minute amounts of an analyte to permeate through the membrane into the sensing layer.

### 1. Working Electrode

In the analyte sensor, the working electrode can be any suitable conductive material, such as carbon, gold, palladium, or platinum. In some embodiments, the working electrode can be a carbon working electrode. The analyte-responsive sensing layer senses a desired analyte (e.g., glucose) and can be continuously or discontinuously disposed on at least a portion of the working electrode. A discontinuous application means that the analyte-responsive sensing layer forms a discrete shape on the working electrode, such as a spot, a line, or a plurality (i.e., an array) of spots and/or lines. The number of spots or lines is not considered to be particularly limited, but can range from about 2 to about 10, from about 3 to about 8, or from about 4 to about 6. In some embodiments, the number of spots or lines can be 2, 3, 4, 5, 6, 7, 8, 9, or 10. In some embodiments, the analyte-responsive sensing layer can be continuous on the working electrode. In some embodiments, the analyte-responsive sensing layer can be discontinuous on the working electrode.

### 2. Sensing Layer

In some embodiments, the working electrode can comprise at least one sensing layer. In some embodiments, the working electrode can comprise one sensing layer. In some embodiments, the working electrode can comprise two sensing layers. In some embodiments, the working electrode can comprise a first sensing layer and a second sensing layer, wherein the analyte for the first sensing layer is different from the analyte for the second sensing layer. In this instance, the first sensing layer and second sensing layer can form an array of multiple spots of each sensing layer, in which some spots sense a first analyte (e.g., glucose) and other spots sense a second analyte different from the first analyte (e.g., ketone, creatinine). Each spot can range in size from about 0.01 to about 1 mm² in diameter.

The total size of the sensing layer or areas (combined area of all spots, layers, or sensing layers) can be from about 0.05 mm² to about 100 mm². In some embodiments, the total size can be about 100 mm² or less, about 75 mm² or less, about 50 mm² or less, about 40 mm² or less, about 30 mm² or less, about 25 mm² or less, about 15 mm² or less, about 10 mm² or less, about 5 mm² or less, about 1 mm² or less, or about 0.1 mm² or less. In some embodiments, the total size of the sensing layer or areas ranges from about 0.05 mm² to about 0.1 mm², from about 0.05 mm² to about 100 mm², from about 0.1 mm² to about 50 mm², from about 0.5 mm² to about 30 mm², from about 1 mm² to about 20 mm², or from about 1 mm² to about 15 mm².

The sensing layer or areas can typically have a thickness that ranges from about 0.1 μm to about 10 μm. For example, each sensing layer can be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically can have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In some embodiments, each sensing layer has a thickness from about 0.1 μm to about 10 μm, from about 0.2 μm to about 8 μm, from about 0.5 μm to about 5 μm, from about 1 μm to about 4 μm, or from about 1 μm about 2 μm.

In some embodiments, a conductive material such as, for example, carbon nanotubes, graphene, or metal nanoparticles, can be combined within the sensing layer or layers to promote rapid attainment of a steady state current. Conductive material can be included in a range from about 0.1% to about 50% by weight (pbw) of the sensing layer (e.g., from about 1 pbw to about 50 pbw, from about 1 pbw to about 10 pbw, or from about 0.1 pbw to about 10 pbw).

### 3. Cofactor Supply

In some embodiments, the analyte sensors of the present disclosure can include a supply of a cofactor in the sensing layer. For example, but not by way of limitation, the present disclosure provides analyte sensors that can include a supply of a cofactor that allows the controlled release of the cofactor over an extended period of the time.

The exact amount of the cofactor supply present within an analyte sensor can vary based on the particular application of the analyte sensor, e.g., which analyte is being detected, the duration of analyte detection, and the conditions under which the detection of the analyte occurs.

In some embodiments, the cofactor is NAD(P). Non-limiting examples of NAD(P) derivatives are disclosed in WO 2007/012494 and WO 1998/033936, the contents of each which are incorporated herein by reference in their entireties. In some embodiments, the present disclosure provides analyte sensors that can include a supply of NAD(P) in the sensing layer that allows the controlled release of NAD(P) or derivative thereof over an extended period of the time. In some embodiments, the supply of NAD(P) can be an internal supply of NAD(P), e.g., an NAD(P) depot, as disclosed in US 2022/0186277, the contents of which are incorporated herein by reference in its entirety.

In some embodiments, the cofactor is FAD (P).

In some embodiments, the amount of NAD(P) present within a sensing layer or an NAD(P) depot can vary depending on the duration of use of the analyte sensor. In some embodiments, NAD(P) can be present in a sensing layer or an NAD(P) depot in an amount from about 0.1 μg to about 900 μg. In some embodiments, from about 0.1 μg to about 900 μg, from about 0.1 μg to about 600 μg, from about 0.1 μg to about 300 μg, from about 0.1 μg to about 100 μg, from about 0.1 μg to about 50 μg, from about 0.1 μg to about 25 μg, from about 0.1 μg to about 10 μg, from about 0.1 μg to about 1 μg, from about 0.1 μg to about 0.5 μg, from about 0.5 μg to about 900 μg, from about 0.5 μg to about 600 μg, from about 0.5 μg to about 300 μg, from about 0.5 μg to about 100 μg, from about 0.5 μg to about 50 μg, from about 0.5 μg to about 25 μg, from about 0.5 μg to about 10 μg, from about 0.5 μg to about 1 μg, from about 1 μg to about 900 μg, from about 1 μg to about 600 μg, from about 1 μg to about 300 μg, from about 1 μg to about 100 μg, from about 1 μg to about 50 μg, from about 1 μg to about 25 μg, from about 1 μg to about 10 μg, from about 10 μg to about 900 μg, from about 10 μg to about 600 μg, from about 10 μg to about 300 μg, from about 10 μg to about 100 μg, from about 10 μg to about 50 μg, from about 10 μg to about 25 μg, from about 25 μg to about 900 μg, from about 25 μg to about 600 μg, from about 25 μg to about 300 μg, from about 25 μg to about 100 μg, from about 25 μg to about 50 μg, from about 50 μg to about 900 μg, from about 50 μg to about 600 μg, from about 50 μg to about 300 μg, from about 50 μg to about 100 μg, from about 100 μg to about 900 μg, from about 100 μg to about 600 μg, from about 100 μg to about 300 μg, from about 300 μg to about 900 μg, from about 300 μg to about 600 μg, or from about 600 μg to about 900 μg of NAD(P) can be present in a sensing layer or an NAD(P) depot. In some embodiments, NAD(P) can be present in a sensing layer or an NAD(P) depot in an amount from about 0.1 μg to about 100 μg.

In some embodiments, the amount of NAD(P) present in the sensing layer or NAD(P) depot can vary depending on the lifetime of the analyte sensor. In some embodiments, the amount of NAD(P) in the sensing layer or NAD(P) depot can allow the analyte sensor to detect an analyte using an NAD(P)-dependent enzyme for at least about 7 days, for at least about 8 days, for at least about 9 days, for at least about 10 days, for at least about 11 days, for at least about 12 days, for at least about 13 days, for at least about 14 days, for at least about 15 days, for at least about 16 days, for at least about 17 days, for at least about 18 days, for at least about 19 days, for at least about 20 days, for at least about 25 days, for at least about 30 days, for at least about 35 days, or for at least about 40 days. In some embodiments, the amount of NAD(P) in the sensing layer or NAD(P) depot can allow the analyte sensor to detect an analyte using an NAD(P)-dependent enzyme for at least about 14 days. In some embodiments, the amount of NAD(P) in the sensing layer or NAD(P) depot can allow the analyte sensor to detect an analyte using an NAD(P)-dependent enzyme for greater than about two weeks, for greater than about three weeks, for greater than about four weeks, for greater than about five weeks, for greater than about six weeks, for greater than about seven weeks, or for greater than about eight weeks.

In some embodiments, the amount of FAD (P) present within a sensing layer can vary depending on the duration of use of the analyte sensor. In some embodiments, FAD (P) can be present in a sensing layer in an amount from about 0.1 μg to about 900 μg. In some embodiments, from about 0.1 μg to about 900 μg, from about 0.1 μg to about 600 μg, from about 0.1 μg to about 300 μg, from about 0.1 μg to about 100 μg, from about 0.1 μg to about 50 μg, from about 0.1 μg to about 25 μg, from about 0.1 μg to about 10 μg, from about 0.1 μg to about 1 μg, from about 0.1 μg to about 0.5 μg, from about 0.5 μg to about 900 μg, from about 0.5 μg to about 600 μg, from about 0.5 μg to about 300 μg, from about 0.5 μg to about 100 μg, from about 0.5 μg to about 50 μg, from about 0.5 μg to about 25 μg, from about 0.5 μg to about 10 μg, from about 0.5 μg to about 1 μg, from about 1 μg to about 900 μg, from about 1 μg to about 600 μg, from about 1 μg to about 300 μg, from about 1 μg to about 100 μg, from about 1 μg to about 50 μg, from about 1 μg to about 25 μg, from about 1 μg to about 10 μg, from about 10 μg to about 900 μg, from about 10 μg to about 600 μg, from about 10 μg to about 300 μg, from about 10 μg to about 100 μg, from about 10 μg to about 50 μg, from about 10 μg to about 25 μg, from about 25 μg to about 900 μg, from about 25 μg to about 600 μg, from about 25 μg to about 300 μg, from about 25 μg to about 100 μg, from about 25 μg to about 50 μg, from about 50 μg to about 900 μg, from about 50 μg to about 600 μg, from about 50 μg to about 300 μg, from about 50 μg to about 100 μg, from about 100 μg to about 900 μg, from about 100 μg to about 600 μg, from about 100 μg to about 300 μg, from about 300 μg to about 900 μg, from about 300 μg to about 600 μg, or from about 600 μg to about 900 μg of FAD (P) can be present in a sensing layer. In some embodiments, FAD (P) can be present in a sensing layer in an amount from about 0.1 μg to about 100 μg.

In some embodiments, the amount of FAD (P) present in the sensing layer can vary depending on the lifetime of the analyte sensor. In some embodiments, the amount of FAD (P) in the sensing layer can allow the analyte sensor to detect an analyte using an FAD (P)-dependent enzyme for at least about 7 days, for at least about 8 days, for at least about 9 days, for at least about 10 days, for at least about 11 days, for at least about 12 days, for at least about 13 days, for at least about 14 days, for at least about 15 days, for at least about 16 days, for at least about 17 days, for at least about 18 days, for at least about 19 days, for at least about 20 days, for at least about 25 days, for at least about 30 days, for at least about 35 days, or for at least about 40 days. In some embodiments, the amount of FAD (P) in the sensing layer can allow the analyte sensor to detect an analyte using an FAD (P)-dependent enzyme for at least about 14 days. In some embodiments, the amount of FAD (P) in the sensing layer can allow the analyte sensor to detect an analyte using an FAD (P)-dependent enzyme for greater than about two weeks, for greater than about three weeks, for greater than about four weeks, for greater than about five weeks, for greater than about six weeks, for greater than about seven weeks, or for greater than about eight weeks.

### 4. Highly Permeable Membrane

Embodiments of the present disclosure relate to systems for improving the performance of one or more components of a sensor by inclusion of a membrane configured to have an analyte permeability that is substantially temperature insensitive, i.e., that does not substantially vary with changes in temperature. As used herein the term “permeability” refers to a physical property of a substance that is related to the rate of diffusion of permeate (e.g., a mobile substance) through a substance (e.g., a solid, a semi-solid, a gel, a hydrogel, or a membrane). Permeability relates to the grade of transmissibility of the substance, i.e., how much permeate diffuses through the substance in a specific time. In some instances, the permeability of a substance depends on the type of permeate, the size of the permeate, the pressure, the temperature, the type of substance, the thickness of the substance, the surface area of the substance, the pore size of the substance, the tortuosity of the substance, the density of the substance, and the like.

The term permeability includes substances that are semi-permeable. Semi-permeability refers to the property of a substance to be permeable only for certain molecules or ions and not for others. For example, a semi-permeable membrane (e.g., a selectively permeable membrane, a partially-permeable membrane, or a differentially-permeable membrane) is a membrane that will only allow certain molecules or ions to pass through it by diffusion. The rate of passage can depend on the pressure, concentration, and temperature of the molecules or solutes on the other side, as well as the permeability of the membrane to each solute.

As used herein, the phrase “highly permeable” means that a greater percentage of an analyte can diffuse across a substrate (e.g., a membrane) over a certain period of time than can diffuse across a comparable substrate (e.g., a membrane).

In some embodiments, at least 50% more analyte can diffuse across a membrane comprising a copolymer of poly(N-vinylimidazole) and poly(N-isopropylacrylamide) over a period of 14 days than can diffuse across a membrane comprising 10Q5 polymer. In some embodiments, at least 75% more analyte can diffuse across a membrane comprising a copolymer of poly(N-vinylimidazole) and poly(N-isopropylacrylamide) over a period of 14 days than can diffuse across a membrane comprising 10Q5 polymer. In some embodiments, at least 100% more analyte can diffuse across a membrane comprising a copolymer of poly(N-vinylimidazole) and poly(N-isopropylacrylamide) over a period of 14 days than can diffuse across a membrane comprising 10Q5 polymer. In some embodiments, at least 150% more analyte can diffuse across a membrane comprising a copolymer of poly(N-vinylimidazole) and poly(N-isopropylacrylamide) over a period of 14 days than can diffuse across a membrane comprising 10Q5 polymer.

In some embodiments, 50% more analyte can diffuse across a membrane comprising a copolymer of poly(N-vinylimidazole) and poly(N-isopropylacrylamide) over a period of 14 days than can diffuse across a membrane comprising a copolymer of poly(4-vinylpyridine-co-N-isopropylacrylamide). In some embodiments, 75% more analyte can diffuse across a membrane comprising a copolymer of poly(N-vinylimidazole) and poly(N-isopropylacrylamide) over a period of 14 days than can diffuse across a membrane comprising a copolymer of poly(4-vinylpyridine-co-N-isopropylacrylamide). In some embodiments, 100% more analyte can diffuse across a membrane comprising a copolymer of poly(N-vinylimidazole) and poly(N-isopropylacrylamide) over a period of 14 days than can diffuse across a membrane comprising a copolymer of poly(4-vinylpyridine-co-N-isopropylacrylamide). In some embodiments, 150% more analyte can diffuse across a membrane comprising a copolymer of poly(N-vinylimidazole) and poly(N-isopropylacrylamide) over a period of 14 days than can diffuse across a membrane comprising a copolymer of poly(4-vinylpyridine-co-N-isopropylacrylamide).

As used herein, the phrase “temperature independent” means that a value does not substantially vary with changes in temperature. For example, the value can vary by about 20% or less, by about 15% or less, by about 10% or less, by about 5% or less, by about 4% or less, by about 3% or less, by about 2% or less, or by about 1% or less as the temperature changes. For example, in some embodiments the membrane retains at least 80%, e.g., at least 85%, e.g., at least 90%, e.g., at least 95%, e.g., at least 96%, e.g., at least 97%, e.g., at least 98%, or at least 99% permeability over a temperature range of from about 0° C. to about 50° C., e.g., from about 15° C. to about 45° C., e.g., from about 20° C. to about 35° C., e.g., from about 25° C. to about 30° C.

In some embodiments, an analyte sensor that comprises a membrane (e.g., highly permeable membrane) can generate signals over a temperature range that are within about 80% or more of each other, within about 85% or more of each other, within about 90% or more of each other, within about 95% or more of each other, within about 96% or more of each other, within about 97% or more of each other, within about 98% or more of each other, or within about 99% or more of each other at a constant analyte concentration.

In some embodiments, an analyte sensor that comprises a membrane (e.g., highly permeable membrane) can generate a signal that varies by no more than about 20%, no more than about 15%, no more than about 10%, no more than about 5%, no more than about 4%, no more than about 3%, no more than about 2%, or no more than about 1% over the temperature range at a constant analyte concentration. In some embodiments, an analyte sensor that comprises a membrane (e.g., highly permeable membrane) can generate a signal that varies by no more than about 5% over the temperature range at a constant analyte concentration.

In some embodiments, an analyte sensor that comprises a membrane (e.g., highly permeable membrane) can generate signals that are substantially temperature independent over a range of temperatures, where the range of temperature can be from about 0° C. to about 50° C., from about 0° C. to about 45° C., from about 0° C. to about 35° C., from about 0° C. to about 25° C., from about 0° C. to about 15° C., from about 15° C. to about 50° C., from about 15° C. to about 45° C., from about 15° C. to about 35° C., from about 15° C. to about 25° C., from about 25° C. to about 50° C., from about 25° C. to about 45° C., from about 25° C. to about 35° C., from about 35° C. to about 50° C., from about 35° C. to about 45° C., or from about 45° C. to about 50° C. In some embodiments, an analyte sensor that comprises a membrane (e.g., highly permeable membrane) can generate signals that are temperature independent from about 25° C. to about 45° C.

In some embodiments, because the analyte sensor is configured to generate signals that are substantially temperature independent, it is not necessary to correct the signals generated by the analyte sensor due to changes in temperature. Thus, analyte sensors having a membrane (e.g., highly permeable membrane) can be used to determine a level of analyte over time without correcting for temperature variation at the sensor. For instance, determining the level of an analyte over a period of time can include monitoring the level of the analyte in a subject in the absence of correcting for temperature variation in the sensor. In addition, because the analyte sensor is configured to generate signals that are substantially temperature independent, in some embodiments, the analyte sensors do not include a temperature measurement device, such as a thermistor.

In some embodiments, an analyte sensor that comprises a membrane (e.g., a highly permeable membrane) can have increased sensitivity to an analyte of interest. As used herein, the “sensitivity” of an analyte sensor can be defined as the ratio between the analyte sensor current level (nA) and the blood analyte level (mM); i.e., nA/mM. Thus, sensitivity can be measured in units of nA/mM.

In some embodiments, an analyte sensor that comprises a membrane (e.g., a highly permeable membrane) can have a sensitivity to an analyte of interest of 100 nA/mM or more, 150 nA/mM or more, 200 nA/mM or more, 250 nA/mM or more, or 300 nA/mM or more. In some embodiments, an analyte sensor that comprises a membrane (e.g., a highly permeable membrane) can have a sensitivity to an analyte of interest from about 0.1 nA/mM to about 300 nA/mM, from about 0.1 nA/mM to about 250 nA/mM, from about 0.1 nA/mM to about 200 nA/mM, from about 0.1 nA/mM to about 150 nA/mM, from about 0.1 nA/mM to about 100 nA/mM, from about 0.1 nA/mM to about 50 nA/mM, from about 0.1 nA/mM to about 25 nA/mM, from about 0.1 nA/mM to about 10 nA/mM, from about 0.1 nA/mM to about 5 nA/mM, from about 0.1 nA/mM to about 2.5 nA/mM, from about 0.1 nA/mM to about 1 nA/mM, from about 1 nA/mM to about 300 nA/mM, from about 1 nA/mM to about 250 nA/mM, from about 1 nA/mM to about 200 nA/mM, from about 1 nA/mM to about 150 nA/mM, from about 1 nA/mM to about 100 nA/mM, from about 1 nA/mM to about 50 nA/mM, from about 1 nA/mM to about 25 nA/mM, from about 1 nA/mM to about 10 nA/mM, from about 1 nA/mM to about 5 nA/mM, from about 1 nA/mM to about 2.5 nA/mM, from about 2.5 nA/mM to about 300 nA/mM, from about 2.5 nA/mM to about 250 nA/mM, from about 2.5 nA/mM to about 200 nA/mM, from about 2.5 nA/mM to about 150 nA/mM, from about 2.5 nA/mM to about 100 nA/mM, from about 2.5 nA/mM to about 50 nA/mM, from about 2.5 nA/mM to about 25 nA/mM, from about 2.5 nA/mM to about 10 nA/mM, from about 2.5 nA/mM to about 5 nA/mM, from about 5 nA/mM to about 300 nA/mM, from about 5 nA/mM to about 250 nA/mM, from about 5 nA/mM to about 200 nA/mM, from about 5 nA/mM to about 150 nA/mM, from about 5 nA/mM to about 100 nA/mM, from about 5 nA/mM to about 50 nA/mM, from about 5 nA/mM to about 25 nA/mM, from about 5 nA/mM to about 10 nA/mM, from about 10 nA/mM to about 300 nA/mM, from about 10 nA/mM to about 250 nA/mM, from about 10 nA/mM to about 200 nA/mM, from about 10 nA/mM to about 150 nA/mM, from about 10 nA/mM to about 100 nA/mM, from about 10 nA/mM to about 50 nA/mM, from about 10 nA/mM to about 25 nA/mM, from about 25 nA/mM to about 300 nA/mM, from about 25 nA/mM to about 250 nA/mM, from about 25 nA/mM to about 200 nA/mM, from about 25 nA/mM to about 150 nA/mM, from about 25 nA/mM to about 100 nA/mM, from about 25 nA/mM to about 50 nA/mM, from about 50 nA/mM to about 300 nA/mM, from about 50 nA/mM to about 250 nA/mM, from about 50 nA/mM to about 200 nA/mM, from about 50 nA/mM to about 150 nA/mM, from about 50 nA/mM to about 100 nA/mM, from about 100 nA/mM to about 300 nA/mM, from about 100 nA/mM to about 250 nA/mM, from about 100 nA/mM to about 200 nA/mM, from about 100 nA/mM to about 150 nA/mM, from about 150 nA/mM to about 300 nA/mM, from about 150 nA/mM to about 250 nA/mM, from about 150 nA/mM to about 200 nA/mM, from about 200 nA/mM to about 300 nA/mM, from about 200 nA/mM to about 250 nA/mM, or from about 250 nA/mM to about 300 nA/mM. In some embodiments, an analyte sensor that comprises a membrane (e.g., a highly permeable membrane) can have a sensitivity to an analyte of interest from about 100 nA/mM to about 300 nA/mM. In some embodiments, an analyte sensor that comprises a membrane (e.g., a highly permeable membrane) can have a sensitivity to an analyte of interest of about 300 nA/mM, about 250 nA/mM, about 200 nA/mM, about 150 nA/mM, about 100 nA/mM, or about 50 nA/mM.

In some embodiments, an analyte sensor that comprises a membrane (e.g., a highly permeable membrane) can have a sensitivity to glucose from about about 150 nA/mM to about 300 nA/mM. In some embodiments, an analyte sensor that comprises a membrane (e.g., a highly permeable membrane) can have a sensitivity to glucose of about 286 nA/mM. In some embodiments, an analyte sensor that comprises a membrane (e.g., a highly permeable membrane) can have a sensitivity to glucose of about 188 nA/mM. In some embodiments, an analyte sensor that comprises a membrane (e.g., a highly permeable membrane) can have a sensitivity to glutamate from about 150 nA/mM to about 200 nA/mM. In some embodiments, an analyte sensor that comprises a membrane (e.g., a highly permeable membrane) can have a sensitivity to glutamate of about 179 nA/mM.

In some embodiments, an analyte sensor that comprises a membrane (e.g., a highly permeable membrane) can have a sensitivity that is 90% or more of the initial sensitivity after 1 day or more, after 2 days or more, after 3 days or more, after 4 days or more, after 5 days or more, after 6 days or more, after 7 days or more, after 10 days or more, after 14 days or more, after 15 days or more, after 1 month or more, after 2 months or more, after 4 months or more, after 6 months or more, after 9 months or more, or after 1 year or more. In some embodiments, an analyte sensor that comprises a membrane (e.g., a highly permeable membrane) can have a sensitivity that is 95% or more of the initial sensitivity after 1 day or more, after 2 days or more, after 3 days or more, after 4 days or more, after 5 days or more, after 6 days or more, after 7 days or more, after 10 days or more, after 14 days or more, after 15 days or more, after 1 month or more, after 2 months or more, after 4 months or more, after 6 months or more, after 9 months or more, or after 1 year or more. In some embodiments, an analyte sensor that comprises a membrane (e.g., a highly permeable membrane) can have a sensitivity that is 97% or more of the initial sensitivity after 1 day or more, after 2 days or more, after 3 days or more, after 4 days or more, after 5 days or more, after 6 days or more, after 7 days or more, after 10 days or more, after 14 days or more, after 15 days or more, after 1 month or more, after 2 months or more, after 4 months or more, after 6 months or more, after 9 months or more, or after 1 year or more.

In some embodiments, therefore, the analyte sensor has a sensitivity that remains at least 80%, e.g., at least 85%, e.g., at least 90%, e.g., at least 95%, e.g., at least 96%, e.g., at least 97%, e.g., at least 98%, or at least 99% constant over a temperature range of from about 0° C. to about 50° C., e.g., from about 15° C. to about 45° C., e.g., from about 20° C. to about 35° C., e.g., from about 25° C. to about 30° C. for at least 1 day, e.g., at least 2 days, e.g., at least 3, 4, 5, 6, 7, 10, 14, or 15 days, at least 1 month, at least 2, 4, 6, or 9 months or at least 1 year.

In some embodiments, the analyte sensor shows an analyte sensitivity that is greater than the analyte sensitivity of an otherwise identical sensor lacking a membrane (e.g., a highly permeable membrane) comprising a copolymer of poly(N-vinylimidazole) and poly(N-isopropylacrylamide). In some embodiments, the analyte sensor shows an analyte sensitivity that is at least 25% greater than the analyte sensitivity of an otherwise identical sensor lacking a membrane (e.g., a highly permeable membrane) comprising a copolymer of poly(N-vinylimidazole) and poly(N-isopropylacrylamide). In some embodiments, the analyte sensor shows an analyte sensitivity that is at least 50% greater than the analyte sensitivity of an otherwise identical sensor lacking a membrane (e.g., a highly permeable membrane) comprising a copolymer of poly(N-vinylimidazole) and poly(N-isopropylacrylamide). In some embodiments, the analyte sensor shows an analyte sensitivity that is at least 75% greater than the analyte sensitivity of an otherwise identical sensor lacking a highly permeable membrane comprising a copolymer of poly(N-vinylimidazole) and poly(N-isopropylacrylamide). In some embodiments, the analyte sensor shows an analyte sensitivity that is at least 100% greater than the analyte sensitivity of an otherwise identical sensor lacking a membrane (e.g., a highly permeable membrane) comprising a copolymer of poly(N-vinylimidazole) and poly(N-isopropylacrylamide).

In some embodiments, the sensing area is overcoated with a membrane as described herein. In some embodiments, at least about 10%, at least about 20%, at least about 30%, at least about 40%, at least about 50%, at least about 60%, at least about 70%, at least about 80%, at least about 90% or at least about 95% of the sensing area is overcoated with the membrane. In some embodiments, the sensing area is entirely overcoated with the membrane. In some embodiments, the sensing layer can be overcoated with a membrane (e.g., a highly permeable membrane). In some embodiments, at least about 10%, at least about 20%, at least about 30%, at least about 40%, at least about 50%, at least about 60%, at least about 70%, at least about 80%, at least about 90% or at least about 95% of the sensing layer can be overcoated with a membrane (e.g., a highly permeable membrane). In some embodiments, the sensing layer can be entirely overcoated with a membrane (e.g., a highly permeable membrane).

The membrane (e.g., highly permeable membrane) can be applied over the sensing layer(s) by placing a droplet or droplets of membrane solution on at least the one or more sensing layers of an analyte sensor, such as by dipping the implantable portion (e.g., sensor tail) into a membrane solution, by spraying the membrane solution on the implantable portion (e.g., sensor tail), by heat pressing or melting the membrane solution, by vapor depositing the membrane solution, by powder coating the membrane solution, or combinations thereof.

Generally, the thickness of the membrane (e.g., highly permeable membrane) can be controlled by the number of different membrane solutions, the concentration of the membrane solution(s), by the number of droplets of the membrane solution(s) applied, by the number of times the implantable portion (e.g., sensor tail) is dipped in the membrane solution(s), by the volume of membrane solution(s) sprayed on the implantable portion (e.g., sensor tail), or by any combination thereof. In some embodiments, the membrane (e.g., highly permeable membrane) can have a thickness ranging from about 0.1 μm to about 1000 μm, from about 1 μm to about 500 μm, from about 10 μm to about 100 μm, or from about 10 μm to about 20 μm. In some embodiments, the membrane (e.g., highly permeable membrane) can have a thickness ranging from about 0.1 μm to about 25 μm, from about 0.1 μm to about 20 μm, from about 0.1 μm to about 15 μm, from about 0.1 μm to about 10 μm, from about 0.1 μm to about 5 μm, from about 0.1 μm to about 1 μm, from about 1 μm to about 25 μm, from about 1 μm to about 20 μm, from about 1 μm to about 15 μm, from about 1 μm to about 10 μm, from about 1 μm to about 5 μm, from about 5 μm to about 25 μm, from about 5 μm to about 20 μm, from about 5 μm to about 15 μm, from about 5 μm to about 10 μm, from about 10 μm to about 25 μm, from about 10 μm to about 20 μm, from about 10 μm to about 15 μm, from about 15 μm to about 25 μm, from about 15 μm to about 20 μm, or from about 20 μm to about 25 μm. In some embodiments, the membrane (e.g., highly permeable membrane) can have a thickness of about 25 μm, about 20 μm, about 19 μm, about 18 μm, about 15 μm, about 10 μm, about 5 μm, about 1 μm, or about 0.1 μm. In some embodiments, the membrane (e.g., highly permeable membrane) can have a thickness of about 18 μm to about 22 μm. For example, but not by way of limitation, a sensor (or working electrode) of the present disclosure can be dipped in a membrane solution, or in each different membrane solution if multiple membrane solutions are used, at least once, at least twice, at least three times, at least four times, or at least five times to obtain the desired membrane (e.g., highly permeable membrane) thickness.

In some embodiments the or each dipping step can be conducted at a relative humidity level of from about 10% to about 80%, such as from about 30% to about 60%, e.g., about 50% to about 55%. In some embodiments the or each dipping step can be conducted at a dip speed (entry and/or exit) of from about 1 mm/sec to about 100 mm/s, e.g., about 10 to about 15 mm/sec. In some embodiments a drying time of from about 1 minute to about 1 hour, e.g., abut 10 minutes to about 20 minutes is allowed between successive dips. In some embodiments the or each dipping step is conducted at a temperature of from about 10° C. to about 40° C., such as from about 15° C. to about 30° C., e.g., from about 20° C. to about 25° C., e.g., about 21° C. In some embodiments the deposited membrane solution is allowed to cure post deposition for a period of from about 1 hour to about 1 week, e.g., from about 12 hours to about 48 hours, e.g., about 24 hours at a temperature of from about 10° C. to about 70° C., such as from about 20° C. to about 60° C., e.g., from about 25° C. to about 55 or 56° C., at a relative humidity of from about 10% to about 80%, such as from about 30% to about 60%.

In some embodiments, the membrane (e.g., highly permeable membrane) can be single-component (i.e. the membrane polymer contains a single type of monomer). In some embodiments, the membrane (e.g., highly permeable membrane) can be multi-component (i.e. the membrane polymer contains two or more different types of monomers). In some embodiments, the membrane (e.g., highly permeable membrane) can comprise 1, 2, 3, 4, 5, 6, 7, or 8 different types of monomers. In some embodiments, the membrane (e.g., highly permeable membrane) can comprises 2 different types of monomers. In some embodiments, the membrane (e.g., highly permeable membrane) can comprise a copolymer.

In some embodiments, the membrane (e.g., the highly permeable membrane) comprises a copolymer of poly(N-vinylimidazole) and poly(N-isopropylacrylamide). In some embodiments, the membrane (e.g., highly permeable membrane) can comprise a copolymer of poly(N-vinylimidazole) and poly(N-isopropylacrylamide) that has a lower critical solution temperature (LCST) in water that is about equal to body temperature. In some embodiments, the membrane (e.g., highly permeable membrane) can comprise a copolymer of poly(N-vinylimidazole) and poly(N-isopropylacrylamide) that has a lower critical solution temperature (LCST) in a buffer that is about equal to body temperature. In some embodiments, the membrane (e.g., highly permeable membrane) can comprise a copolymer of poly(N-vinylimidazole) and poly(N-isopropylacrylamide) that has a lower critical solution temperature (LCST) in phosphate buffered saline that is about equal to body temperature. The LCST is the critical temperature below which the components of a mixture are miscible. The LCST can depend on a number of factors including pressure (e.g., increasing the pressure can increase the LCST), degree of polymerization, polydispersity (e.g., the distribution of molar mass in the polymer), the branching of the polymer, and the like. Raising the temperature above its LCST can result in phase separation (e.g., one or more of the polymers can solidify or crystalize), which can result in a decrease in the rate of diffusion for the membrane. In some embodiments, this decrease in the rate of diffusion for the membrane can substantially offset the increase in the rate of diffusion due to increasing the temperature, such that the membrane has substantially the same rate of diffusion to solutes (e.g., glucose or glutamate) over a temperature range of interest. In some embodiments, the temperature range of interest includes normal body temperature for a human. In some embodiments, the temperature range of interest for a membrane (e.g., highly permeable membrane) comprising a copolymer of poly(N-vinylimidazole) and poly(N-isopropylacrylamide) can be from about 25° C. to about 60° C., from about 25° C. to about 55° C., from about 25° C. to about 45° C., from about 25° C. to about 35° C., from about 35° C. to about 60° C., from about 35° C. to about 55° C., from about 35° C. to about 45° C., from about 45° C. to about 60° C., from about 45° C. to about 55° C., or from about 55° C. to about 60° C. in water. In some embodiments, the temperature range of interest for a membrane (e.g., highly permeable membrane) comprising a copolymer of poly(N-vinylimidazole) and poly(N-isopropylacrylamide) can be from about 25° C. to about 60° C., from about 25° C. to about 55° C., from about 25° C. to about 45° C., from about 25° C. to about 35° C., from about 35° C. to about 60° C., from about 35° C. to about 55° C., from about 35° C. to about 45° C., from about 45° C. to about 60° C., from about 45° C. to about 55° C., or from about 55° C. to about 60° C. in phosphate buffered saline. In some embodiments, the temperature range of interest for a membrane (e.g., highly permeable membrane) comprising a copolymer of poly(N-vinylimidazole) and poly(N-isopropylacrylamide) can be from about 25° C. to about 45° C. in water. In some embodiments, the temperature range of interest for a membrane (e.g., highly permeable membrane) comprising a copolymer of poly(N-vinylimidazole) and poly(N-isopropylacrylamide) can be from 22° C. to 47° C. in water. In some embodiments, the temperature range of interest for a membrane (e.g., highly permeable membrane) comprising a copolymer of poly(N-vinylimidazole) and poly(N-isopropylacrylamide) can be from about 22° C. to about 42° C. in phosphate buffered saline.

In some embodiments, the membrane (e.g., the highly permeable membrane) comprises a copolymer of poly(N-vinylimidazole) and poly(N-isopropylacrylamide) that has a lower critical solution temperature (LCST) in water or buffer (e.g., phosphate buffered saline) of from about 20° C. to about 60° C., e.g., from about 20° C. to about 50° C., e.g., from about 22° C. to about 47° C., e.g., from about 25° C. to about 45° C., or from about 22° C. to about 42° C.

In some embodiments, the membrane (e.g., highly permeable membrane) can comprise a copolymer of poly(N-isopropylacrylamide) and poly(N-vinylimidazole).

In some embodiments, the copolymers have alternating monomer subunits. In some embodiments, the copolymers can be block copolymers, which include two or more homopolymer subunits linked by covalent bonds. A copolymer of the present disclosure includes a block copolymer. In some embodiments, the copolymer can be a random copolymer.

In some embodiments, the membrane (e.g., highly permeable membrane) comprises a polymer of Formula (I):

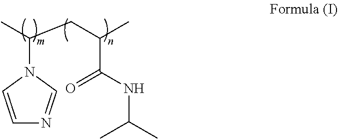

wherein:
m is an integer from 30 to 50; and
n is an integer from 50 to 70.

As used herein, the phrase “number average molecular weight” is defined as the total weight of the polymer sample divided by the total number of molecules in the polymer sample.

In some embodiments, the copolymer can include N-vinylimidazole monomer (i.e., 1-vinylimidazole) in a mole percent (mole %) of at least about 3%, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total copolymer. In some embodiments, the copolymer can include N-vinylimidazole monomer in a mole percent of at least about 80% of the total copolymer. In some embodiments, the copolymer can include N-vinylimidazole monomer in a mole percent of at least about 70% of the total copolymer. In some embodiments, the copolymer can include N-vinylimidazole monomer in a mole percent of at least about 65% of the total copolymer. In some embodiments, the copolymer can include N-vinylimidazole monomer in a mole percent of at least about 60% of the total copolymer. In some embodiments, the copolymer can include N-vinylimidazole monomer in a mole percent of at least about 55% of the total copolymer. In some embodiments, the copolymer can include N-vinylimidazole monomer in a mole percent of at least about 50% of the total copolymer. In some embodiments, the copolymer can include N-vinylimidazole monomer in a mole percent of at least about 45% of the total copolymer. In some embodiments, the copolymer can include N-vinylimidazole monomer in a mole percent of at least about 40% of the total copolymer. In some embodiments, the copolymer can include N-vinylimidazole monomer in a mole percent of at least about 35% of the total copolymer. In some embodiments, the copolymer can include N-vinylimidazole monomer in a mole percent of at least about 30% of the total copolymer. In some embodiments, the copolymer can include N-vinylimidazole in a mole percent of at least about 25% of the total copolymer. In some embodiments, the copolymer can include N-vinylimidazole in a mole percent from about 30% to about 80% of the total copolymer. In some embodiments, the copolymer can include N-vinylimidazole in a mole percent from about 40% to about 80% of the total copolymer. In some embodiments, the copolymer can include N-vinylimidazole in a mole percent from about 40% to about 70% of the total copolymer.

In some embodiments, the copolymer can include N-isopropylacrylamide monomer in a mole percent of at least about 3%, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total copolymer. In some embodiments, the copolymer can include N-isopropylacrylamide monomer in a mole percent of at least about 20% of the total copolymer. In some embodiments, the copolymer can include N-isopropylacrylamide monomer in a mole percent of at least about 25% of the total copolymer. In some embodiments, the copolymer can include N-isopropylacrylamide monomer in a mole percent of at least about 30% of the total copolymer. In some embodiments, the copolymer can include N-isopropylacrylamide monomer in a mole percent of at least about 35% of the total copolymer. In some embodiments, the copolymer can include N-isopropylacrylamide monomer in a mole percent of at least about 40% of the total copolymer. In some embodiments, the copolymer can include N-isopropylacrylamide monomer in a mole percent of at least about 45% of the total copolymer. In some embodiments, the copolymer can include N-isopropylacrylamide monomer in a mole percent of at least about 50% of the total copolymer. In some embodiments, the copolymer can include N-isopropylacrylamide monomer in a mole percent of at least about 55% of the total copolymer. In some embodiments, the copolymer can include N-isopropylacrylamide monomer in a mole percent of at least about 60% of the total copolymer. In some embodiments, the copolymer can include N-isopropylacrylamide monomer in a mole percent of at least about 65% of the total copolymer. In some embodiments, the copolymer can include N-isopropylacrylamide monomer in a mole percent of at least about 70%. In some embodiments, the copolymer can include N-isopropylacrylamide monomer in a mole percent of at least about 75%. In some embodiments, the copolymer can include N-isopropylacrylamide monomer in a mole percent of at least about 80%. In some embodiments, the copolymer can include N-isopropylacrylamide monomer in a mole percent from about 10% to about 80% of the total copolymer. In some embodiments, the copolymer can include N-isopropylacrylamide monomer in a mole percent from about 20% to about 80% of the total copolymer. In some embodiments, the copolymer can include N-isopropylacrylamide monomer in a mole percent from about 30% w/w to about 80% w/w of the total copolymer. In some embodiments, the copolymer can include N-isopropylacrylamide monomer in amount from about 40% w/w to about 80% w/w of the total copolymer. In some embodiments, the copolymer can include N-isopropylacrylamide monomer in a mole percent from about 50% to about 80% of the total copolymer. In some embodiments, the copolymer can include N-isopropylacrylamide monomer in amount from about 30% w/w to about 50% w/w of the total copolymer. In some embodiments, the copolymer can include N-isopropylacrylamide monomer in amount from about 30% w/w to about 60% w/w of the total copolymer. In some embodiments, the copolymer can include N-isopropylacrylamide monomer in amount from about 20% w/w to about 70% w/w of the total copolymer. In some embodiments, the copolymer can include N-isopropylacrylamide monomer in amount from about 30% w/w to about 70% w/w of the total copolymer. In some embodiments, the copolymer can include N-isopropylacrylamide monomer in amount from about 40% w/w to about 70% w/w of the total copolymer. In some embodiments, the copolymer can include N-isopropylacrylamide monomer in a mole percent from about 50% to about 70% of the total copolymer. In some embodiments, the copolymer can include N-isopropylacrylamide monomer in amount from about 30% w/w to about 65% w/w of the total copolymer. In some embodiments, the copolymer can include N-isopropylacrylamide monomer in a mole percent from about 55% to about 65% of the total copolymer. In some embodiments, the copolymer can include N-isopropylacrylamide monomer in a mole percent of about 60% of the total copolymer.

In some embodiments, the membrane (e.g., highly permeable membrane) can comprise a poly(N-vinylimidazole) having a number average molecular weight from about 2800 to about 4800. In some embodiments, the membrane (e.g., highly permeable membrane) can comprise a poly(N-vinylimidazole) having a number average molecular weight from about 2800 to about 4800, from about 2800 to about 4400, from about 2800 to about 4000, from about 2800 to about 3600, from about 2800 to about 3200, from about 3200 to about 4800, from about 3200 to about 4400, from about 3200 to about 4000, from about 3200 to about 3600, from about 3600 to about 4800, from about 3600 to about 4400, from about 3600 to about 4000, from about 4000 to about 4800, from about 4000 to about 4400, or from about 4400 to about 4800.

In some embodiments, the membrane (e.g., highly permeable membrane) can comprise a poly(N-isopropylacrylamide) having a number average molecular weight from about 5600 to about 8000. In some embodiments, the membrane (e.g., highly permeable membrane) can comprise a poly(N-isopropylacrylamide) having a number average molecular weight from about 5600 to about 8000, from about 5600 to about 7600, from about 5600 to about 7200, from about 5600 to about 6800, from about 5600 to about 6400, from about 5600 to about 6000, from about 6000 to about 8000, from about 6000 to about 7600, from about 6000 to about 7200, from about 6000 to about 6800, from about 6000 to about 6400, from about 6400 to about 8000, from about 6400 to about 7600, from about 6400 to about 7200, from about 6400 to about 6800, from about 6800 to about 8000, from about 6800 to about 7600, from about 6800 to about 7200, from about 7200 to about 8000, from about 7200 to about 7600, or from about 7600 to about 8000.

In some embodiments, the membrane (e.g., highly permeable membrane) can comprise a copolymer with a poly(N-vinylimidazole) block having a number average molecular weight from about 2800 to about 4800 and a poly(N-isopropylacrylamide) block having a number average molecular weight from about 5600 to about 8000.

In some embodiments, the membrane (e.g., highly permeable membrane) can comprise a copolymer with a poly(N-vinylimidazole) block having a number average molecular weight from about 3300 to about 4200 and a poly(N-isopropylacrylamide) block having a number average molecular weight from about 6200 to about 7400.

In some embodiments, the membrane (e.g., highly permeable membrane) can comprise a copolymer with a poly(N-vinylimidazole) block having a number average molecular weight of about 3800 and a poly(N-isopropylacrylamide) block having a number average molecular weight of about 6800.

In some embodiments, the membrane (e.g., highly permeable membrane) can comprise a membrane polymer crosslinked with a crosslinking agent disclosed herein.

In some embodiments, the copolymer described herein is provided (e.g., is used in the deposition of the membrane on the working electrode, e.g., on the sensing layer of the working electrode) in a buffered solution. In some embodiments, the copolymer is provided in a buffered solution comprising from about 10% to about 95% v/v (e.g., from about 50% to about 90%, e.g., from about 70% to about 80%, e.g., about 80%) ethanol and from about 5% to about 90% (e.g., from about 10% to about 50%, e.g., from about 20% to about 30%, e.g about 20%) of an aqueous buffer. In some embodiments, the aqueous buffer is buffered to a pH of from about 6 to about 9, e.g., from about pH 7 to about pH 8.5, e.g., about pH 8. In some embodiments, the aqueous buffer comprises one or more buffering agents, such as HEPES, at a concentration of from about 1 mM to about 100 mM, e.g., about 10 mM in the aqueous solution. In some embodiments, the copolymer is provided in a solution comprising about 80% ethanol and about 20% v/v of about 10 mM HEPES at about pH 8.

In some embodiments, the disclosure provides an analyte sensor comprising a first working electrode, a first sensing layer disposed upon a surface of the first working electrode, and a membrane (e.g., a highly permeable membrane) covering at least a part of the first sensing layer; wherein:
the membrane has a thickness of from about 0.1 μm to about 1000 μm, such as from about 1 μm to about 500 μm, e.g., from about 10 μm to about 100 μm, e.g., from about 10 μm to about 20 μm or from about 18 to about 22 μm;
the membrane comprises a copolymer of poly(N-vinylimidazole) and poly(N-isopropylacrylamide); in some embodiments the copolymer can have a lower critical solution temperature (LCST) in water or buffer (e.g., phosphate buffered saline) of from about 20° C. to about 60° C., e.g., from about 20° C. to about 50° C., e.g., from about 22° C. to about 47° C., e.g., from about 25° C. to about 45° C., or from about 22° C. to about 42° C.;
the copolymer comprises from about 30 mol % to about 80 mol % N-vinylimidazole (e.g., from about 40 mol % to about 70 mol % e.g., about 50 mol % to about 60 mol %); and from about 20 mol % to about 70 mol % N-isopropylacrylamide (e.g., from about 30 mol % to about 60 mol %, e.g., from about 40 mol % to about 50 mol %) (wherein the mol % of N-vinylimidazole and the mol % of N-isopropylacrylamide cannot exceed 100 mol %, e.g., wherein the mol % of N-vinylimidazole and the mol % of N-isopropylacrylamide total 100
the copolymer comprises a poly(N-vinylimidazole) block having a number average molecular weight from about 2800 to about 4800, e.g., from about 3300 to about 4200 e.g., about 3800; and a poly(N-isopropylacrylamide) block having a number average molecular weight from about 5600 to about 8000, e.g., from about 6200 to about 7400; e.g., about 6800.

### 5. Enzymes

The sensors of the present disclosure include one or more enzymes for detecting one or more analytes in at least one sensing layer. Suitable enzymes for use in a sensor of the present disclosure can include a NAD(P)-dependent enzyme or an NAD(P)-independent enzyme. For example, an NAD(P)-dependent enzyme for use in the present disclosure can be used for detecting glucose, ketones, lactate, oxygen, hemoglobin A1C, albumin, alcohol, alkaline phosphatase, alanine transaminase, aspartate aminotransferase, bilirubin, blood, urea, nitrogen, calcium, carbon dioxide, chloride, creatinine, hematocrit, lactate, magnesium, oxygen, pH, phosphorus, potassium, sodium, total protein, uric acid, etc. In some embodiments, the analyte to be detected using an NAD(P)-dependent enzyme can be glucose, lactate, ketones, creatinine, alcohol, e.g., ethanol, or the like. In some embodiments, a sensing layer can include multiple enzymes, e.g., an enzyme system, that are collectively responsive to the analyte.

In some embodiments, the enzymes for use in a sensor of the present disclosure can include an enzyme that is NAD(P)-independent. In some embodiments, the NAD(P)-independent enzyme for use in present disclosure can be used for detecting glucose or glutamate.

In some embodiments, the sensing layer of a presently disclosed analyte sensor can include at least one NAD(P)-dependent enzyme. In some embodiments, the sensing layer of a presently disclosed analyte sensor can include two or more NAD(P)-dependent enzymes. In some embodiments, the analyte sensor of the present disclosure can include two sensing layers that each include at least one NAD(P)-dependent enzyme. Alternatively, an analyte sensor of the present disclosure in some embodiments can include two sensing layers, where only one sensing layer includes an NAD(P)-dependent enzyme. Non-limiting examples of NAD(P)-dependent enzymes are disclosed in Vidal et al., Biochimica et Biophysica Acta-Proteins and Proteomics 1866 (2): 327-347 (2018) (see Tables 1-2), the contents of which are incorporated by reference in its entirety.

In some embodiments, an analyte sensor of the present disclosure can include one or more internal supplies of NAD(P) for an NAD(P)-dependent enzyme included in one or more sensing layers of the analyte sensor.

In some embodiments, a sensing layer can include an NAD(P)-dependent dehydrogenase. Non-limiting examples of NAD(P)-dependent dehydrogenases include glucose dehydrogenase (EC.1.1.1.47), lactate dehydrogenase (EC1.1.1.27 and EC1.1.1.28), malate dehydrogenase (EC1.1.1.37), glycerol dehydrogenase (EC1.1.1.6), alcohol dehydrogenase (EC1.1.1.1), alpha-hydroxybutyrate dehydrogenase, sorbitol dehydrogenase, amino acid dehydrogenase such as L-amino acid dehydrogenase (EC1.4.1.5), diaphorase (EC1.8.1.4), and combinations thereof.

In some embodiments, the NAD(P)-dependent dehydrogenase can include diaphorase, glucose dehydrogenase, alcohol dehydrogenase, lactate dehydrogenase, and β-hydroxybutyrate dehydrogenase. In some embodiments, the enzyme system can include two or more NAD(P)-dependent dehydrogenases, e.g., a first NAD(P)-dependent dehydrogenase and diaphorase. For example, but not by way of limitation, the NAD(P)-dependent dehydrogenase can convert the analyte and oxidized nicotinamide adenine dinucleotide (NAD⁺) into an oxidized analyte and reduced nicotinamide adenine dinucleotide (NADH), respectively. The enzyme cofactors NAD⁺ and NADH aid in promoting the concerted enzymatic reactions disclosed herein. The NADH can then undergo reduction under diaphorase mediation, with the electrons transferred during this process providing the basis for analyte detection at the working electrode.

In some embodiments, an analyte sensor of the present disclosure can include a glucose-responsive sensing layer, a ketones-responsive sensing layer, a lactate-responsive sensing layer, a creatinine-responsive sensing layer, an alcohol-responsive sensing layer, or any combination thereof. In some embodiments, a glucose-responsive sensing layer can include one or more NAD(P)-dependent enzymes for detecting glucose. In some embodiments, a ketones-responsive sensing layer can include one or more NAD(P)-dependent enzymes for detecting ketones. In some embodiments, a lactate-responsive sensing layer can include one or more NAD(P)-dependent enzymes for detecting lactate. In some embodiments, a creatinine-responsive sensing layer can include one or more NAD(P)-dependent enzymes for detecting creatinine. In some embodiments, an alcohol-responsive sensing layer can include one or more NAD(P)-dependent enzymes for detecting alcohol. In some embodiments, a sensing layer can include an enzyme system comprising two or more enzymes that are collectively responsive to the analyte. For example, but not by way of limitation, a ketones-responsive sensing layer can include an enzyme system comprising at least one NAD(P)-dependent enzyme.

In some embodiments, an analyte sensor disclosed herein can include at least one sensing layer that includes one or more NAD(P)-dependent enzymes, as disclosed herein, for detecting an analyte. Alternatively, an analyte sensor disclosed herein can include two or more sensing layers, with each sensing layer containing one or more enzymes, e.g., where one of the sensing layers includes one or more NAD(P)-dependent enzymes. For example, but not by way of limitation, an analyte sensor of the present disclosure can include a first sensing layer that comprises a first enzyme (or enzyme system) for use in detecting a first analyte and a second sensing layer that includes a second enzyme (or second enzyme system) for detecting a second analyte, where one of the first sensing layer or second sensing layer can include an NAD(P)-dependent enzyme.

In some embodiments, the enzymes for use in a sensor of the present disclosure can include an enzyme that is a FAD (P)-dependent enzyme. In some embodiments, the enzymes for use in a sensor of the present disclosure can include a FAD (P)-dependent glucose oxidase.

In some embodiments, the sensing layer can include by weight from about 10% to about 80%, e.g., from about 15% to about 75%, from about 20% to about 70%, from about 25% to about 65%, or from about 30% to about 60%, of one or more enzymes disclosed herein.

In some embodiments, the sensing layer can further include a stabilizer, e.g., for stabilizing the enzyme. For example, but not by way of limitation, the stabilizer can be a protein (e.g., an albumin (e.g., a serum albumin)). Non-limiting examples of serum albumins include bovine serum albumin and human serum albumin. In some embodiments, the stabilizer can be a human serum albumin. In some embodiments, the stabilizer can be a bovine serum albumin. In some embodiments, the stabilizer can be catalase. In some embodiments, the sensing layer can include a weight ratio of stabilizer to the one or more enzymes present in the sensing layer, from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In some embodiments, the sensing layer can include a weight ratio of stabilizer to the one or more enzymes present in the sensing layer, from about 2:1 to about 1:2. In some embodiments, the sensing layer can include by weight from about 10% to about 50%, e.g., from about 15% to about 45%, from about 20% to about 40%, from about 20% to about 35%, or from about 20% to about 30% of the stabilizer. In some embodiments, the sensing layer can include from about 15% to about 35% of the stabilizer by weight.

In some embodiments, the sensing layer can further include a cofactor for one or more enzymes present in the sensing layer. In some embodiments, the cofactor can be NAD(P). In some embodiments, the cofactor can be a cofactor different from NAD(P). In some embodiments, the sensing layer can include a weight ratio of cofactor to enzyme from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2, or from about 2:1 to about 1:2. In some embodiments, the sensing layer can include a weight ratio of cofactor to enzyme from about 2:1 to about 1:2. In some embodiments, the sensing layer can include by weight from about 10% to about 50%, e.g., from about 15% to about 45%, from about 20% to about 40%, from about 20% to about 35%, or from about 20% to about 30% of the cofactor. In some embodiments, the sensing layer can include from about 15% to about 35% by weight of the cofactor.

In some embodiments, an analyte sensor of the present disclosure can include an implantable portion (e.g., sensor tail) comprising at least one working electrode, and a sensing layer disposed upon the surface of the working electrode, where the sensing layer includes at least one NAD(P)-independent enzyme. In some embodiments, an analyte sensor of the present disclosure can include an implantable portion (e.g., sensor tail) comprising a substrate, at least one working electrode, and a sensing layer disposed upon the surface of the working electrode, where the sensing layer includes at least one NAD(P)-independent enzyme. In some embodiments, the NAD(P)-independent enzyme can be glucose oxidase or glutamate oxidase. For example, but not by way of limitation, a sensor of the present disclosure can include an implantable portion (e.g., sensor tail) comprising at least one working electrode and a sensing layer disposed upon the surface of the working electrode, where the sensing layer includes an enzyme system comprising glucose oxidase or glutamate oxidase.

In some embodiments, a sensor of the present disclosure can include an implantable portion (e.g., sensor tail) comprising at least one working electrode and a glucose-responsive sensing layer disposed upon the surface of the working electrode, where the glucose-responsive sensing layer includes an enzyme system comprising an NAD(P)-independent oxidase, e.g., glucose oxidase.

In some embodiments, a sensor of the present disclosure can include an implantable portion (e.g., sensor tail) comprising at least one working electrode and a glutamate-responsive sensing layer disposed upon the surface of the working electrode, where the glutamate-responsive sensing layer includes an enzyme system comprising an NAD(P)-independent oxidase, e.g., glutamate oxidase.

In some embodiments, an analyte sensor of the present disclosure can include a second sensing layer, e.g., for detecting an analyte different from the analyte detected by the first sensing layer. In some embodiments, the second sensing layer can be disposed upon the same working electrode as the first sensing layer or on a second working electrode. In some embodiments, the second sensing layer can be a ketones-responsive sensing layer, a lactate-responsive sensing layer, a creatinine-responsive sensing layer, or an alcohol-responsive sensing layer.

In some embodiments, an analyte sensor can include two working electrodes, e.g., a first sensing layer disposed on a first working electrode and a second sensing layer disposed on a second working electrode. For example, but not by way of limitation, an analyte sensor disclosed herein can feature a first sensing layer disposed on a first working electrode and a second sensing layer disposed upon the surface of a different working electrode, e.g., second working electrode, where one of the sensing layers can include an NAD(P)-independent enzyme. In some embodiments, the second sensing layer can be configured to detect a different analyte or the same analyte detected by first sensing layer. In some embodiments, such analyte sensors can include an implantable portion (e.g., sensor tail) with a first working electrode and a second working electrode, a first sensing layer disposed upon a surface of the first working electrode and a second sensing layer disposed upon a surface of the second working electrode, where one of the sensing layers can include an NAD(P)-independent enzyme.

In some embodiments, when the sensor is configured to detect two or more analytes using two working electrodes, detection of each analyte can include applying a potential to each working electrode separately, such that separate signals are obtained from each analyte. The signal obtained from each analyte can then be correlated to an analyte concentration through use of a calibration curve or function, or by employing a lookup table. In some embodiments, correlation of the analyte signal to an analyte concentration can be conducted through use of a processor.

In some analyte sensor configurations, the first sensing layer and the second sensing layer can be disposed upon a single working electrode. For example, but not by way of limitation, an analyte sensor disclosed herein can feature a first sensing layer and a second sensing layer disposed upon the surface of a single working electrode, where one of the sensing layers includes an NAD(P)-independent enzyme. In some embodiments, a first signal can be obtained from the first sensing layer, e.g., at a low potential, and a second signal containing a signal contribution from both sensing layers can be obtained at a higher potential. Subtraction of the first signal from the second signal can then allow the signal contribution arising from the second analyte to be determined. The signal contribution from each analyte can then be correlated to an analyte concentration in a similar manner to that described for sensor configurations having multiple working electrodes. In some embodiments, when a glutamate-responsive sensing layer and a second sensing layer configured to detect a different analyte, e.g., a glucose-responsive sensing layer, are arranged upon a single working electrode in this manner, one of the sensing layers can be configured such that it can be interrogated separately to facilitate detection of each analyte. For example, either the glutamate-responsive sensing layer or glucose-responsive sensing layer can produce a signal independently of the other sensing layer.

It is also to be appreciated that the sensitivity (output current) of the analyte sensors toward each analyte can be varied by changing the coverage (area or size) of the sensing layers, the area ratio of the sensing layers with respect to one another, or the identity, thickness and/or composition of a mass transport limiting membrane overcoating the sensing layers. Variation of these parameters can be conducted readily by one having ordinary skill in the art once granted the benefit of the disclosure herein.

### 6. Redox Mediators

In some embodiments, an analyte sensor comprising a membrane (e.g., a highly permeable membrane) can include an electron transfer agent. For example, but not by way of limitation, one or more sensing layers of an analyte sensor can include an electron transfer agent. In some embodiments, an analyte sensor can include one sensing layer that includes an electron transfer agent and a second sensing layer that does not include an electron transfer agent. In some embodiments, the presence of an electron transfer agent in a sensing layer can depend on the enzyme or enzyme system used to detect the analyte and/or the composition of the working electrode. Alternatively, an analyte sensor can include two sensing layers, where both sensing layers include an electron transfer agent.

Suitable electron transfer agents can facilitate conveyance of electrons to the adjacent working electrode after an analyte undergoes an enzymatic oxidation-reduction reaction within the corresponding sensing layer, thereby generating a current that is indicative of the presence of that particular analyte. The amount of current generated is proportional to the quantity of analyte that is present. In some embodiments, suitable electron transfer agents can include electroreducible and electrooxidizable ions, complexes or molecules (e.g., quinones) having oxidation-reduction potentials that are a few hundred millivolts above or below the oxidation-reduction potential of the standard calomel electrode (SCE). In some embodiments, the redox mediators can include osmium complexes and other transition metal complexes, such as those described in U.S. Pat. Nos. 6,134,461 and 6,605,200, which are incorporated herein by reference in their entirety. Additional examples of suitable redox mediators include those described in U.S. Pat. Nos. 6,736,957, 7,501,053 and 7,754,093, the disclosures of each of which are also incorporated herein by reference in their entirety. Other examples of suitable redox mediators include metal compounds or complexes of ruthenium, osmium, iron (e.g., polyvinylferrocene or hexacyanoferrate), or cobalt, including metallocene compounds thereof, for example. Suitable ligands for the metal complexes can also include, for example, bidentate or higher denticity ligands such as, for example, bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate, or higher denticity ligands can be present in a metal complex, e.g., osmium complex, to achieve a full coordination sphere.

In some embodiments, electron transfer agents disclosed herein can comprise suitable functionality to promote covalent bonding to a polymer (also referred to herein as a polymeric backbone) within the sensing layers as discussed further below. For example, but not by way of limitation, an electron transfer agent for use in the present disclosure can include a polymer-bound electron transfer agent. Suitable non-limiting examples of polymer-bound electron transfer agents include those described in U.S. Pat. Nos. 8,444,834, 8,268,143 and 6,605,201, the disclosures of which are incorporated herein by reference in their entirety. In some embodiments, the electron transfer agent is a bidentate osmium complex bound to a polymer described herein, e.g., a polymeric backbone described below. In some embodiments, the polymer-bound electron transfer agent shown in FIG. 3 of U.S. Pat. No. 8,444,834 can be used in a sensor of the present disclosure.

In some embodiments of the present disclosure, an analyte sensor can include at least one working electrode and at least one sensing layer disposed upon the surface of the working electrode, where the sensing layer can be overcoated with a membrane (e.g., a highly permeable membrane), and at least one redox mediator, e.g., an osmium complex. In some embodiments, the sensing layer includes an enzyme system comprising glutamate oxidase or glucose oxidase and a redox mediator, e.g., an osmium complex.

### 7. Polymeric Backbone

In some embodiments, one or more sensing layers for promoting analyte detection can include a polymer to which an enzyme and/or redox mediator is covalently bound. Any suitable polymeric backbone can be present in the sensing layer for facilitating detection of an analyte through covalent bonding of the enzyme and/or redox mediator thereto. Non-limiting examples of suitable polymers within the sensing layer include polyvinylpyridines, e.g., poly(4-vinylpyridine) and/or poly(2-vinylpyridine), and polyvinylimidazoles, e.g., poly(N-vinylimidazole) and poly(1-vinylimidazole), or a copolymer thereof, for example, in which quaternized pyridine groups serve as a point of attachment for the redox mediator or enzyme thereto. Illustrative copolymers that can be suitable for inclusion in the sensing layers include those containing monomer units such as styrene, acrylamide, methacrylamide, or acrylonitrile, for example. In some embodiments, polymers that can be present in a sensing layer include a polyurethane or a copolymer thereof, and/or polyvinylpyrrolidone. In some embodiments, polymers that can be present in the sensing layer include, but are not limited to, those described in U.S. Pat. No. 6,605,200, the contents of which are incorporated herein by reference in their entirety, such as poly(acrylic acid), styrene/maleic anhydride copolymer, methylvinylether/maleic anhydride copolymer (GANTREZ polymer), poly(vinylbenzylchloride), poly(allylamine), polylysine, poly(4-vinylpyridine) quaternized with carboxypentyl groups, and poly(sodium 4-styrene sulfonate). In some embodiments where the analyte sensor includes two sensing layers, the polymer within each sensing layer can be the same or different.

In some embodiments, the polymer can be polyvinylpyridine or a copolymer thereof. In some embodiments, the polymer can be a co-polymer of vinylpyridine and styrene.

In some embodiments, when an enzyme system with multiple enzymes is present in a given sensing layer, all of the multiple enzymes can be covalently bonded to the polymer. In some embodiments, only a subset of the multiple enzymes are covalently bonded to the polymer. For example, and not by the way of limitation, one or more enzymes within an enzyme system can be covalently bonded to the polymer and at least one enzyme can be non-covalently associated with the polymer, such that the non-covalently bonded enzyme is physically retained within the polymer. In some embodiments, the NAD(P)-dependent enzyme can be covalently bonded to the polymer. Alternatively, the NAD(P)-dependent enzyme can be non-covalently associated with the polymer. In some embodiments, the NAD(P)-dependent dehydrogenase and the diaphorase can be covalently bonded to a polymer within a sensing layer of the disclosed analyte sensors. In some embodiments, the NAD(P)-dependent dehydrogenase can be covalently bonded to the polymer and diaphorase can be non-covalently associated with the polymer. Alternatively, diaphorase can be covalently bonded to the polymer and the NAD(P)-dependent dehydrogenase can be non-covalently associated with the polymer.

In some embodiments, when a stabilizer is present in a sensing layer, one or more enzymes within the area can be covalently bonded to the stabilizer. For example, and not by the way of limitation, one or more enzymes within an enzyme system, e.g., one or more NAD(P)-dependent enzymes, can be covalently bonded to the stabilizer, e.g., albumin, present in the sensing layer.

In some particular embodiments, covalent bonding of the one or more enzymes and/or redox mediators to the polymer and/or stabilizer in a given sensing layer can take place via crosslinking introduced by a suitable crosslinking agent. In some embodiments, crosslinking of the polymer to the one or more enzymes and/or redox mediators can reduce the occurrence of delamination of the enzyme compositions from the electrode. Suitable crosslinking agents can include one or more crosslinkable functionalities such as, but not limited to, vinyl, alkoxy, acetoxy, enoxy, oxime, amino, hydroxyl, cyano, halo, acrylate, epoxide, and isocyanato groups. In some embodiments, the crosslinking agent can comprise one or more, two or more, three or more, or four or more epoxide groups. For example, but not by way of limitation, a crosslinker for use in the present disclosure can include mono-, di-, tri- and tetra-ethylene oxides. In some embodiments, crosslinking agents for reaction with free amino groups in the enzyme (e.g., with the free side chain amine in lysine) can include crosslinking agents such as, for example, polyethylene glycol dibutyl ethers, polypropylene glycol dimethyl ethers, polyalkylene glycol allyl methyl ethers, polyethylene glycol diglycidyl ether (PEGDGE), or other polyepoxides, cyanuric chloride, N-hydroxysuccinimide, imidoesters, epichlorohydrin, or derivatized variants thereof. In some embodiments, the crosslinking agent can be PEGDGE, e.g., having an average molecular weight (Mn) from about 200 to 1,000, e.g., about 400. In some embodiments, the crosslinking agent can be PEGDGE 400. In some embodiments, the crosslinking agent can be glutaraldehyde. Suitable crosslinking agents for reaction with free carboxylic acid groups in the enzyme can include, for example, carbodiimides. In some embodiments, the crosslinking agent can be polyethylene glycol diglycidyl ether. In some embodiments, the crosslinking of the enzyme to the polymer can generally be intermolecular. In some embodiments, the crosslinking of the enzyme to the polymer can generally be intramolecular.

In some embodiments, an analyte sensor of the present disclosure comprises a sensing layer as described herein comprising one or more enzymes (e.g., one or more NAD(P)-dependent enzymes, e.g., one or more NAD(P)-dependent dehydrogenases or oxidases as described herein; and/or one or more NAD(P)-independent enzymes, e.g., glucose oxidase); a stabilizer (e.g., a protein, e.g., an albumin) and a cofactor (e.g., NAD(P) or a derivative thereof); wherein the sensing layer comprises from about 10% to about 80% (e.g., from about 30% to about 60%) by weight of the enzyme; from about 10% to about 50% (e.g., from about 15% to about 35%) by weight of the stabilizer; and from about 10% to about 50% (e.g., from about 15% to about 35%) by weight of the cofactor. In some embodiments the sensing layer comprises an electron transfer agent (e.g., an osmium complex as described herein bound to a polymeric backbone as described herein). In some embodiments the enzyme(s) and/or the stabilizer are bonded to the polymeric backbone of the electron transfer agent, e.g., by being crosslinked to the polymeric backbone, e.g., by crosslinking with one or more crosslinking agents as described herein, e.g., a PEGDGE. In some embodiments the sensing layer is at least partially overcoated by a membrane (e.g., a highly permeable membrane) as described herein.

### 8. Mass Transport Limiting Membrane

In some embodiments, an analyte sensor comprising a membrane (e.g., a highly permeable membrane) can further comprise a mass transport limiting membrane permeable to an analyte that overcoats at least one sensing layer, e.g., a first sensing layer and/or a second sensing layer. In some embodiments, the mass transport limiting membrane overcoats one or more of the sensing layers of an analyte sensor. In some embodiments, a membrane (e.g., a highly permeable membrane) can overcoat the first sensing layer and a mass transport limiting membrane can overcoat the second sensing layer. In some embodiments, a membrane (e.g., a highly permeable membrane) can overcoat more than one sensing layer. In some embodiments, a membrane (e.g., a highly permeable membrane) can overcoat one of the sensing layers and a mass transport limiting membrane can overcoat both the first and second sensing layers.

In some embodiments, a mass transport limiting membrane overcoating a sensing layer can improve biocompatibility. A mass transport limiting membrane can act as a diffusion-limiting barrier to reduce the rate of mass transport of the analyte, e.g., glucose, an alcohol, a ketone, lactate or β-hydroxybutyrate, when the sensor is in use. For example, but not by way of limitation, limiting access of an analyte, e.g., an alcohol, to the sensing layer with a mass transport limiting membrane can aid in avoiding sensor overload (saturation), thereby improving detection performance and accuracy. In some embodiments, the mass transport limiting membrane can limit the flux of an analyte to the electrode in an electrochemical sensor so that the sensor is linearly responsive over a large range of analyte concentrations.

In some embodiments, the mass transport limiting membrane can have a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the mass transport limiting membrane can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm.

In some embodiments, the mass transport limiting membrane can be formed by depositing a mass transport limiting membrane solution upon a surface, for example by dipping, and allowing the membrane solution to dry. In some embodiments, the sensor can be dipped in the mass transport limiting membrane solution more than once. For example, but not by way of limitation, a sensor (or working electrode) of the present disclosure can be dipped in an mass transport limiting membrane solution at least twice, at least three times, at least four times, or at least five times to obtain the desired mass transport limiting membrane thickness.

In some embodiments, the mass transport limiting membrane can be single-component (contain a single membrane polymer). Alternatively, the mass transport limiting membrane can be multi-component (contain two or more different membrane polymers). In some embodiments, the mass transport limiting membrane can include two or more layers, e.g., a bilayer or trilayer membrane. In some embodiments, each layer can comprise a different polymer or the same polymer at different concentrations or thicknesses. In some embodiments, the first sensing layer can be covered by a membrane (e.g., a highly permeable membrane), and the second sensing layer can be covered by a single mass transport limiting membrane. In some embodiments, the first sensing layer can be covered by a membrane (e.g., a highly permeable membrane), and the second sensing layer can be covered by a multilayered mass transport limiting membrane. In some embodiments, the first sensing layer can be covered by a membrane (e.g., a highly permeable membrane) and the second sensing layer can be covered by a highly permeable membrane. In some embodiments, the first sensing layer can be covered by a membrane (e.g., a highly permeable membrane) and the first sensing layer and the second sensing layer can be covered by a single mass transport limiting membrane.

In some embodiments, a mass transport limiting membrane can include a polyvinylpyridine (e.g., poly(4-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In some embodiments, the mass transport limiting membrane for use in the present disclosure, e.g., a single-component membrane, can include a polyvinylpyridine (e.g., poly(4-vinylpyridine) and/or poly(2-vinylpyridine)). In some embodiments, a mass transport limiting membrane for use in the present disclosure, e.g., a single-component membrane, can include poly(4-vinylpyridine). In some embodiments, a mass transport limiting membrane for use in the present disclosure, e.g., a single-component membrane, can include a copolymer of vinylpyridine and styrene. In some embodiments, the mass transport limiting membrane can comprise a polyvinylpyridine-co-styrene copolymer. For example, but not by way of limitation, a polyvinylpyridine-co-styrene copolymer for use in the present disclosure can include a polyvinylpyridine-co-styrene copolymer in which a portion of the pyridine nitrogen atoms were functionalized with a non-crosslinked polyethylene glycol tail and a portion of the pyridine nitrogen atoms were functionalized with an alkylsulfonic acid group. In some embodiments, a derivatized polyvinylpyridine-co-styrene copolymer for use as a membrane polymer can be the 10Q5 polymer as described in U.S. Pat. No. 8,761,857, the contents of which are incorporated by reference in their entirety. In some embodiments, the polyvinylpyridine-based polymer can have a molecular weight from about 50 Da to about 500 kDa.

In some embodiments, the mass transport limiting membrane can comprise polymers such as, but not limited to, poly(styrene co-maleic anhydride), dodecylamine and poly(propylene glycol)-block-polyethylene glycol)-block-poly(propylene glycol) (2-aminopropyl ether) crosslinked with poly(propylene glycol)-block-poly(ethylene glycol)-block-poly(propylene glycol) bis(2-aminopropyl ether); poly(N-isopropylacrylamide); a copolymer of poly(ethylene oxide) and poly(propylene oxide); or a combination thereof.

In some embodiments, the mass transport limiting membrane can include a polyurethane membrane that includes both hydrophilic and hydrophobic regions. In some embodiments, a hydrophobic polymer component can be a polyurethane, a polyurethane urea or poly(ether-urethane-urea). In some embodiments, a polyurethane is a polymer produced by the condensation reaction of a diisocyanate and a difunctional hydroxyl-containing material. In some embodiments, a polyurethane urea is a polymer produced by the condensation reaction of a diisocyanate and a difunctional amine-containing material. In some embodiments, diisocyanates for use herein can include aliphatic diisocyanates, e.g., containing from about 4 to about 8 methylene units, or diisocyanates containing cycloaliphatic moieties. Additional non-limiting examples of polymers that can be used for the generation of a mass transport limiting membrane of the presently disclosed sensor include vinyl polymers, polyethers, polyesters, polyamides, inorganic polymers (e.g., polysiloxanes and polycarbosiloxanes), natural polymers (e.g., cellulosic and protein based materials) and mixtures (e.g., admixtures or layered structures) or combinations thereof. In some embodiments, the hydrophilic polymer component can be polyethylene oxide and/or polyethylene glycol. In some embodiments, the hydrophilic polymer component can be a polyurethane copolymer. For example, but not by way of limitation, a hydrophobic-hydrophilic copolymer component for use in the present disclosure can be a polyurethane polymer that comprises about 10% to about 50%, e.g., 20%, hydrophilic polyethylene oxide.

In some embodiments, the mass transport limiting membrane can include a silicone polymer/hydrophobic-hydrophilic polymer blend. In some embodiments, the hydrophobic-hydrophilic polymer for use in the blend can be any suitable hydrophobic-hydrophilic polymer such as, but not limited to, polyvinylpyrrolidone, polyhydroxyethyl methacrylate, polyvinylalcohol, polyacrylic acid, polyethers such as polyethylene glycol or polypropylene oxide, and copolymers thereof, including, for example, di-block, tri-block, alternating, random, comb, star, dendritic, and graft copolymers. In some embodiments, the hydrophobic-hydrophilic polymer can be a copolymer of poly(ethylene oxide) (PEO) and poly(propylene oxide) (PPO). Non-limiting examples of PEO and PPO copolymers include PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof. In some embodiments, the copolymers can be substituted with hydroxy substituents.

In some embodiments, hydrophilic or hydrophobic modifiers can be used to “fine-tune” the permeability of the resulting membrane to an analyte of interest. In some embodiments, hydrophilic modifiers such as poly(ethylene) glycol, hydroxyl or polyhydroxyl modifiers and the like, and any combinations thereof, can be used to enhance the biocompatibility of the polymer or the resulting mass transport limiting membrane.

In some embodiments where multiple sensing layers are present, the mass transport limiting membrane can overcoat each sensing layer, including the option of overcoating a sensing layer coated with a membrane (e.g., a highly permeable membrane), which can be achieved by dip coating operations to produce a mass transport limiting membrane portion upon a the membrane (e.g., highly permeable membrane).

In some embodiments where multiple sensing layers are present, a separate mass transport limiting membrane can overcoat each sensing layer, including a sensing layer already overcoated with a membrane (e.g., highly permeable membrane). For example, but not by way of limitation, a mass transport limiting membrane can be disposed on the first sensing layer, e.g., an alcohol-responsive sensing layer, and a separate, second mass transport limiting membrane can overcoat the second sensing layer, e.g., a glucose-responsive sensing layer. In some embodiments, the two mass transport limiting membranes can be spatially separated and do not overlap each other. In some embodiments, the first mass transport limiting membrane does not overlap the second mass transport limiting membrane and the second mass transport limiting membrane does not overlap the first mass transport limiting membrane. In some embodiments, the first mass transport limiting membrane comprises different polymers than the second mass transport limiting membrane. Alternatively, the first mass transport limiting membrane comprises the same polymers as the second mass transport limiting membrane. In some embodiments, the first mass transport limiting membrane comprises the same polymers as the second mass transport limiting membrane but comprises different crosslinking agents. In some embodiments, polydimethylsiloxane (PDMS) can be incorporated in any of the mass transport limiting membranes disclosed herein.

In some embodiments when a first sensing layer and a second sensing layer configured for assaying different analytes are disposed on separate working electrodes, the mass transport limiting membrane can have differing permeability values for the first analyte and the second analyte. For example, but not by way of limitation, the mass transport limiting membrane overcoating at least one of the sensing layers can include an admixture of a first membrane polymer and a second membrane polymer or a bilayer of the first membrane polymer and the second membrane polymer. A homogeneous membrane can overcoat the sensing layer not overcoated with the admixture or the bilayer, wherein the homogeneous membrane includes only one of the first membrane polymer or the second membrane polymer. Advantageously, the architectures of the analyte sensors disclosed herein readily allow a continuous membrane having a homogenous membrane portion to be disposed upon a first sensing layer and a multi-component membrane portion to be disposed upon a second sensing layer of the analyte sensors, thereby equalizing the permeability values for each analyte concurrently to afford improved sensitivity and detection accuracy. Continuous membrane deposition can take place through sequential dip coating operations in particular embodiments.

In some embodiments, the mass transport limiting membrane can comprise a membrane polymer crosslinked with a crosslinking agent disclosed herein. In some embodiments where there are two mass transport limiting membranes, e.g., a first mass transport limiting membrane and a second mass transport limiting membrane, each membrane can be crosslinked with a different crosslinking agent. For example, but not by way of limitation, the crosslinking agent can result in a membrane that is more restrictive to diffusion of certain compounds, e.g., analytes within the membrane, or less restrictive to diffusion of certain compounds, e.g., by affecting the size of the pores within the membrane. For example, but not by way of limitation, in a sensor that is configured to detect alcohol and glucose, the mass transport limiting membrane overcoating the alcohol-responsive area can have a pore size that restricts the diffusion of compounds larger than alcohol, e.g., glucose, through the membrane.

In some embodiments, where an analyte sensor comprises a membrane (e.g., a highly permeable membrane) and a mass transport limiting membrane, each membrane can be crosslinked with a different crosslinking agent. In some embodiments, where the analyte sensor comprises a membrane (e.g., highly permeable membrane) and a mass transport limiting membrane, each membrane can be crosslinked with the same crosslinking agent.

In some embodiments, crosslinking agents for use in the present disclosure can include polyepoxides, carbodiimide, cyanuric chloride, triglycidyl glycerol, N-hydroxysuccinimide, imidoesters, epichlorohydrin or derivatized variants thereof. In some embodiments, a membrane polymer overcoating one or more sensing layers can be crosslinked with a branched crosslinker, e.g., which can decrease the amount of extractables obtainable from the mass transport limiting membrane. Non-limiting examples of a branched crosslinker include branched glycidyl ether crosslinkers, e.g., including branched glycidyl ether crosslinkers that include two or three or more crosslinkable groups. In some embodiments, the branched crosslinker can include two or more crosslinkable groups, such as polyethylene glycol diglycidyl ether. In some embodiments, the branched crosslinker can include three or more crosslinkable groups, such as polyethylene glycol tetraglycidyl ether. In some embodiments, the membrane polymer can include polyvinylpyridine or a copolymer of vinylpyridine and styrene crosslinked with a branched glycidyl ether crosslinker including two or three crosslinkable groups, such as polyethylene glycol tetraglycidyl ether or polyethylene glycol diglycidyl ether. In some embodiments, the epoxide groups of a polyepoxides, e.g., polyethylene glycol tetraglycidyl ether or polyethylene glycol diglycidyl ether, can form a covalent bond with pyridine or an imidazole via epoxide ring opening resulting in a hydroxyalkyl group bridging a body of the crosslinker to the heterocycle of the membrane polymer.

In some embodiments, the crosslinking agent can be polyethylene glycol diglycidyl ether (PEGDGE). In some embodiments, the PEGDGE used to promote crosslinking (e.g., intermolecular crosslinking) between two or more membrane polymer backbones can exhibit a broad range of suitable molecular weights. In some embodiments, the molecular weight of the PEGDGE can range from about 100 g/mol to about 5,000 g/mol. The number of ethylene glycol repeat units in each arm of the PEGDGE can be the same or different, and can typically vary over a range within a given sample to afford an average molecular weight. In some embodiments, the PEGDGE for use in the present disclosure has a number average molecular weight (Mn) from about 200 to 1,000, e.g., about 400. In some embodiments, the crosslinking agent can be PEGDGE 400.

In some embodiments, the polyethylene glycol tetraglycidyl ether used to promote crosslinking (e.g., intermolecular crosslinking) between two or more membrane polymer backbones can exhibit a broad range of suitable molecular weights. Up to four polymer backbones can be crosslinked with a single molecule of the polyethylene glycol tetraglycidyl ether crosslinker. The number of ethylene glycol repeat units in each arm of the polyethylene glycol tetraglycidyl ether can be the same or different, and can typically vary over a range within a given sample to afford an average molecular weight.

In some embodiments, the membrane (e.g., a highly permeable membrane) can include a crosslinking agent in a weight/volume (w/v) percent from about 0.5% to about 30% of the total weight/volume of the membrane (e.g., a highly permeable membrane). In some embodiments, the membrane (e.g., a highly permeable membrane) can include a crosslinking agent in a w/v percent from about 0.5% to about 30%, from about 0.5% to about 20%, from about 0.5% to about 10%, from about 0.5% to about 5%, from about 0.5% to about 4%, from about 0.5% to about 2%, from about 0.5 to about 1%, from about 1% to about 20%, from about 1% to about 10%, from about 1% to about 5%, from about 1% to about 4%, from about 1% to about 2%, from about 2% to about 30%, from about 2% to about 20%, from about 2% to about 10%, from about 2% to about 5%, from about 2% to about 4%, from about 4% to about 30%, from about 4% to about 20%, from about 4% to about 10%, from about 4% to about 5%, from about 5% to about 30%, from about 5% to about 20%, from about 5% to about 10%, from about 10% to about 30%, from about 10% to about 20%, or from about 20% to about 30% of the total weight/volume of the membrane (e.g., highly permeable membrane).

In some embodiments, the membrane (e.g., highly permeable membrane) can include polyethylene glycol diglycidyl ether (PEGDGE) 400 in a weight/volume (w/v) percent from about 0.5% to about 30% of the total weight/volume of the membrane (e.g., highly permeable membrane). In some embodiments, the membrane (e.g., highly permeable membrane) can include PEGDGE 400 in a w/v percent from about 0.5% to about 30%, from about 0.5% to about 20%, from about 0.5% to about 10%, from about 0.5% to about 5%, from about 0.5% to about 4%, from about 0.5% to about 2%, from about 0.5 to about 1%, from about 1% to about 20%, from about 1% to about 10%, from about 1% to about 5%, from about 1% to about 4%, from about 1% to about 2%, from about 2% to about 30%, from about 2% to about 20%, from about 2% to about 10%, from about 2% to about 5%, from about 2% to about 4%, from about 4% to about 30%, from about 4% to about 20%, from about 4% to about 10%, from about 4% to about 5%, from about 5% to about 30%, from about 5% to about 20%, from about 5% to about 10%, from about 10% to about 30%, from about 10% to about 20%, or from about 20% to about 30% of the total weight/volume of the highly permeable membrane. In some embodiments, the membrane (e.g., highly permeable membrane) can include a crosslinking agent in a weight-volume (w/v) percent of about 4% of the total weight/volume of the membrane (e.g., highly permeable membrane).

In some embodiments, the membrane (e.g., highly permeable membrane) can include a ratio (w/v) of crosslinking agent to copolymer of poly(N-vinylimidazole) and poly(N-isopropylacrylamide) from about 40:1 to about 1:40. In some embodiments, the membrane (e.g., highly permeable membrane) can include a ratio (w/v) of crosslinking agent to copolymer of poly(N-vinylimidazole) and poly(N-isopropylacrylamide) from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2, or from about 2:1 to about 1:2. In some embodiments, the membrane (e.g., highly permeable membrane) can include a ratio (w/v) of crosslinking agent to copolymer of poly(N-vinylimidazole) and poly(N-isopropylacrylamide) from about 10:1 to about 1:10. In some embodiments, the membrane (e.g., highly permeable membrane) can include a ratio (w/v) of crosslinking agent to copolymer of poly(N-vinylimidazole) and poly(N-isopropylacrylamide) from about 6:1 to about 1:6. In some embodiments, the membrane (e.g., highly permeable membrane) can include a ratio (w/v) of crosslinking agent to copolymer of poly(N-vinylimidazole) and poly(N-isopropylacrylamide) from about 1:2 to about 1:10. In some embodiments, the membrane (e.g., highly permeable membrane) can include a ratio (w/v) of crosslinking agent to copolymer of about 1:6.

In some embodiments, the membrane (e.g., highly permeable membrane) can include a ratio (w/v) of polyethylene glycol diglycidyl ether (PEGDGE) 400 to copolymer of poly(N-vinylimidazole) and poly(N-isopropylacrylamide) from about 40:1 to about 1:40. In some embodiments, the membrane (e.g., highly permeable membrane) can include a ratio (w/v) of PEGDGE 400 to copolymer of poly(N-vinylimidazole) and poly(N-isopropylacrylamide) from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2, or from about 2:1 to about 1:2. In some embodiments, the membrane (e.g., highly permeable membrane) can include a ratio (w/v) of PEGDGE 400 to copolymer of poly(N-vinylimidazole) and poly(N-isopropylacrylamide) from about 1:100 to about 1:40. In some embodiments, the membrane (e.g., highly permeable membrane) can include a ratio (w/v) of crosslinking agent to copolymer from about 1:100 to about 1:10. In some embodiments, the membrane (e.g., highly permeable membrane) can include a ratio (w/v) of PEGDGE 400 to copolymer of poly(N-vinylimidazole) and poly(N-isopropylacrylamide) from about 1:100 to about 1:1. In some embodiments, the membrane (e.g., highly permeable membrane) can include a ratio (w/v) of PEGDGE 400 to copolymer of poly(N-vinylimidazole) and poly(N-isopropylacrylamide) of about 1:40 to about 1:20.

In some embodiments the crosslinking agent is provided (e.g., is incorporated in the membrane solution used to deposit the membrane on the working electrode, e.g., on the sensing layer of the working electrode) in a buffered solution. In some embodiments the crosslinking agent is provided in an buffered solution comprising from about 10% to about 95% v/v (e.g., from about 50% to about 90%, e.g., from about 70% to about 80%, e.g., about 80%) ethanol and from about 5% to about 90% (e.g., from about 10% to about 50%, e.g., from about 20% to about 30%, e.g about 20%) of an aqueous buffer. In some embodiments the aqueous buffer is buffered to a pH of from about 6 to about 9, e.g., from about pH 7 to about pH 8.5, e.g., about pH 8. In some embodiments the aqueous buffer comprises one or more buffering agents, such as HEPES, at a concentration of from about 1 mM to about 100 mM, e.g., about 10 mM in the aqueous solution. For example, in some embodiments the crosslinking agent is provided in a solution comprising about 80% ethanol and about 20% v/v of about 10 mM HEPES at about pH 8.

Accordingly, in some embodiments the disclosure provides an analyte sensor comprising a first working electrode, a first sensing layer disposed upon a surface of the first working electrode, and a membrane (e.g., a highly permeable membrane) covering at least a part of the first sensing layer; wherein:
the membrane (e.g., a highly permeable membrane) has a thickness of from about 0.1 μm to about 1000 μm, such as from about 1 μm to about 500 μm, e.g., from about 10 μm to about 100 μm, e.g., from about 10 μm to about 20 μm or from about 18 to about 22 μm;
the membrane (e.g., a highly permeable membrane) comprises a copolymer of poly(N-vinylimidazole) and poly(N-isopropylacrylamide); in some embodiments the copolymer can have a lower critical solution temperature (LCST) in water or buffer (e.g., phosphate buffered saline) of from about 20° C. to about 60° C., e.g., from about 20° C. to about 50° C., e.g., from about 22° C. to about 47° C., e.g., from about 25° C. to about 45° C., or from about 22° C. to about 42° C.;
the copolymer comprises from about 30 mol % to about 80 mol % N-vinylimidazole (e.g., from about 40 mol % to about 70 mol % e.g., about 50 mol % to about 60 mol %); and from about 20 mol % to about 70 mol % N-isopropylacrylamide (e.g., from about 30 mol % to about 60 mol %, e.g., from about 40 mol % to about 50 mol %) (wherein the mol % of N-vinylimidazole and the mol % of N-isopropylacrylamide cannot exceed 100 mol %, e.g., wherein the mol % of N-vinylimidazole and the mol % of N-isopropylacrylamide total 100
the copolymer comprises a poly(N-vinylimidazole) block having a number average molecular weight from about 2800 to about 4800, e.g., from about 3300 to about 4200 e.g., about 3800; and a poly(N-isopropylacrylamide) block having a number average molecular weight from about 5600 to about 8000, e.g., from about 6200 to about 7400; e.g., about 6800;
the membrane (e.g., a highly permeable membrane) is cross-linked with a crosslinking agent, wherein the crosslinking agent is polyethylene glycol diglycidyl ether (PEGDGE) having a molecular weight of from about 100 g/mol to about 5,000 g/mol (e.g., having an average molecular weight (Mn) of from about 200 to 1,000, e.g., the crosslinking agent may be PEGDGE 400); wherein the crosslinking agent is present in the membrane in a weight/volume ratio to the copolymer of from about 1:100 to about 1:10.

In some embodiments, the sensing layer comprises:
one or more enzymes (e.g., one or more NAD(P)-dependent enzymes, e.g., one or more NAD(P)-dependent dehydrogenases or oxidases as described herein; and/or one or more NAD(P)-independent enzymes, e.g., glucose oxidase);
an optional stabilizer (e.g., a protein, e.g., an albumin);
one or more cofactors (e.g., NAD(P) or a derivative thereof); and
an electron transfer agent (e.g., an osmium complex as described herein bound to a polymeric backbone as described herein);
optionally wherein the enzyme(s) and/or the stabilizer (if present) are bonded to the polymeric backbone of the electron transfer agent, e.g., by being crosslinked to the polymeric backbone, e.g., by crosslinking with one or more crosslinking agents as described herein, e.g., a PEGDGE.

### 9. Interference Domain

In some embodiments, the sensor of the present disclosure, e.g., an implantable portion thereof (e.g., a sensor tail), can further comprise an interference domain. In some embodiments, the interference domain can include a polymer domain that restricts the flow of one or more interferents, e.g., to the surface of the working electrode. In some embodiments, the interference domain can function as a molecular sieve that allows analytes and other substances that are to be measured by the working electrode to pass through, while preventing passage of other substances such as interferents. In some embodiments, the interferents can affect the signal obtained at the working electrode. Non-limiting examples of interferents include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In some embodiments, the interference domain can be located between the working electrode and one or more sensing layers, e.g., alcohol-responsive sensing layer. In some embodiments, non-limiting examples of polymers that can be used in the interference domain include polyurethanes, polymers having pendant ionic groups, and polymers having controlled pore size. In some embodiments, the interference domain is formed from one or more cellulosic derivatives. Non-limiting examples of cellulosic derivatives include polymers such as cellulose acetate, cellulose acetate butyrate, 2-hydroxyethyl cellulose, cellulose acetate phthalate, cellulose acetate propionate, cellulose acetate trimellitate, and the like.

In some embodiments, the interference domain can be part of the mass transport limiting membrane and not a separate membrane.

In some embodiments, the interference domain can include a thin, hydrophobic membrane that is non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen, and/or ascorbic acid.

In some embodiments, the interference domain can be deposited directly onto the working electrode, e.g., onto the surface of the working electrode. In some embodiments, the interference domain has a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the interference domain can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm. In some embodiments, the sensor can be dipped in the interference domain solution more than once. For example, but not by way of limitation, a sensor (or working electrode) of the present disclosure can be dipped in an interference domain solution at least once, at least twice, at least three times, at least four times, or at least five times to obtain the desired interference domain thickness.

### 10. Manufacturing

The present disclosure further provides methods for manufacturing the presently disclosed analyte sensors that includes one or more sensing layers and one or more working electrodes.

In some embodiments, the method can include depositing one or more enzymes on a working electrode. In some embodiments, an enzyme composition can include one or more enzymes (e.g., as described herein), a crosslinking agent, e.g., polyethylene glycol diglycidyl ether (e.g., as described herein), and/or a redox mediator (e.g., as described herein). In some embodiments, the enzyme composition can be deposited onto the surface of a working electrode as one large application which covers the desired portion of the working electrode or in the form of an array of a plurality of enzyme compositions, e.g., spaced apart from each other, to generate one or more sensing layers for detecting one or more analytes. In some embodiments, the method can further include curing the enzyme composition.

In some embodiments, the method includes depositing one or more NAD(P)-dependent enzymes, e.g., an NAD(P)-dependent dehydrogenase, on a working electrode. In some embodiments, the enzyme composition can include one or more additional enzymes, e.g., diaphorase, a crosslinking agent, e.g., polyethylene glycol diglycidyl ether, and/or a redox mediator. In some embodiments, the enzyme composition can be deposited onto the surface of a working electrode as one large application which covers the desired portion of the working electrode or in the form of an array of a plurality of enzyme compositions, e.g., spaced apart from each other, to generate one or more sensing layers for detecting one or more analytes. In some embodiments, the method can further include curing the enzyme composition.

In some embodiments, the method can further include adding a membrane (e.g., a highly permeable membrane) on top of the cured enzyme composition.

In some embodiments, the sensing layer composition and the membrane (e.g., highly permeable membrane) can be prepared as solutions that dry or cure to solidify after deposition. Therefore, in some embodiments, all layers can be deposited in an automated fashion using small-volume liquid handling or similar techniques for high-throughput sensor fabrication.

In some embodiments, the method can further include adding a membrane composition on top of the cured sensing layer and/or around the entire sensor. In some embodiments, the membrane composition is a membrane (e.g., a highly permeable membrane), a mass transport limiting membrane, or a combination. In some embodiments, the method can include curing the membrane composition.

### IV. ANALYTE MONITORING

The present disclosure further provides methods of using the analyte sensors disclosed herein to detect an analyte in vivo. In some embodiments, the present disclosure provides methods for detecting one or more analytes, e.g., one analyte or two analytes. For example, but not by way of limitation, the present disclosure provides methods for detecting one or more analytes including glucose, ketones, lactate, oxygen, hemoglobin A1C, albumin, alcohol, alkaline phosphatase, alanine transaminase, aspartate aminotransferase, bilirubin, blood, urea, nitrogen, calcium, carbon dioxide, chloride, creatinine, hematocrit, lactate, magnesium, oxygen, pH, phosphorus, potassium, sodium, total protein, and/or uric acid using one or more NAD(P)-dependent or NAD(P)-independent enzymes. In some embodiments, the analyte can be ketones, alcohol, glucose, and/or lactate using one or more NAD(P)-dependent enzymes. In some embodiments, the analyte can be glucose or glutamate using one or more NAD(P)-independent enzymes. For example, but not by way of limitation, the present disclosure provides methods for detecting one or more ketones. In some embodiments, the present disclosure provides methods for detecting glucose. In some embodiments, the present disclosure provides methods for detecting creatinine. In some embodiments, the present disclosure provides methods for detecting lactate. In some embodiments, the present disclosure provides methods for detecting alcohol. In some embodiments, the present disclosure provides methods for detecting glutamate.

In some embodiments, the present disclosure provides methods for monitoring in vivo levels of an analyte over time with analyte sensors that include one or more NAD(P)-dependent enzymes or NAD(P)-independent enzymes. Generally, monitoring the in vivo concentration of an analyte in a fluid of the body of a subject includes inserting at least partially under a skin surface an in vivo analyte sensor as disclosed herein, contacting the monitored fluid (interstitial, blood, dermal, and the like) with the inserted sensor and generating a sensor signal at the working electrode. The presence and/or concentration of the analyte detected by the analyte sensor can be displayed, stored, forwarded, and/or otherwise processed. A variety of approaches can be employed to determine the concentration of analyte (e.g., glucose, an alcohol, a ketone, and/or lactate) with the disclosed sensors. In some embodiments, monitoring the concentration of analyte using the sensor signal can be performed by coulometric, amperometric, voltammetric, potentiometric, or any other convenient electrochemical detection technique.

In some embodiments, the analyte sensors comprising a membrane (e.g., a highly permeable membrane) display increased stability. In some embodiments, the analyte sensors comprising a membrane (e.g., a highly permeable membrane), wherein the membrane (e.g., highly permeable membrane) comprises a copolymer of poly(N-vinylimidazole) and poly(N-isopropylacrylamide), exhibit less than a 20% decrease (signal drop) in current over a period of 12 days. In some embodiments, the analyte sensors comprising a membrane (e.g., a highly permeable membrane), wherein the membrane (e.g., highly permeable membrane) comprises a copolymer of poly(N-vinylimidazole) and poly(N-isopropylacrylamide), exhibit less than a 20% decrease, less than a 15% decrease, less than a 10% decrease, or less than a 5% decrease in current over a period of 12 days. In some embodiments, the analyte sensors comprising a membrane (e.g., a highly permeable membrane), wherein the membrane (e.g., highly permeable membrane) comprises a copolymer of poly(N-vinylimidazole) and poly(N-isopropylacrylamide), exhibit less than a 15% decrease in current over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a membrane (e.g., a highly permeable membrane), wherein the membrane (e.g., highly permeable membrane) comprises a copolymer of poly(N-vinylimidazole) and poly(N-isopropylacrylamide), exhibit less than a 10% decrease in current over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than a 20% decrease in current over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days.

In some embodiments, the analyte sensors comprising a membrane (e.g., highly permeable membrane) exhibit about a 5% decrease in current over a period of 15 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than 20% delamination over a period of 12 days. In some embodiments, the analyte sensors comprising a membrane (e.g., highly permeable membrane) exhibit less than 20% delamination, less than a 15% delamination, less than 10% delamination, or less than 5% delamination over a period of 12 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than 20% delamination over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than 15% delamination over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than 10% delamination over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a membrane (e.g., highly permeable membrane), wherein the membrane (e.g., highly permeable membrane) comprises a copolymer of poly(N-vinylimidazole) and poly(N-isopropylacrylamide), exhibit less than 5% delamination over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days.

In some embodiments, a method for detecting an analyte includes:
(i) applying a potential to a first working electrode of an analyte sensor, wherein the analyte sensor comprises:
   (a) a first working electrode;
   (b) a first sensing layer disposed upon a surface of the first working electrode; and
   (c) a highly permeable membrane that overcoats at least a part of the first sensing layer and that is permeable to a first analyte; wherein the highly permeable membrane comprises a copolymer of poly(N-vinylimidazole) and poly(N-isopropylacrylamide);
wherein the analyte sensor shows a sensitivity of at least 100 nA/mM to the first analyte;
(ii) obtaining a first signal at or above an oxidation-reduction potential of the first sensing layer, the first signal being proportional to a concentration of a first analyte in a fluid contacting the first sensing layer; and fluid.
(iii) correlating the first signal to the concentration of the first analyte in the

In some embodiments, a method for detecting glucose includes:
(i) applying a potential to a first working electrode of an analyte sensor, wherein the analyte sensor comprises:
   (a) a first working electrode;
   (b) a glucose-responsive sensing layer disposed upon a surface of the first working electrode; and
   (c) a highly permeable membrane that overcoats at least a part of the glucose-responsive sensing layer and that is permeable to glucose; wherein the highly permeable membrane comprises a copolymer of poly(N-vinylimidazole) and poly(N-isopropylacrylamide);
wherein the analyte sensor shows a sensitivity of at least 100 nA/mM to glucose;
(ii) obtaining a first signal at or above an oxidation-reduction potential of the first sensing layer, the first signal being proportional to a concentration of glucose in a fluid contacting the first sensing layer; and
(iii) correlating the first signal to the concentration of glucose in the fluid.

In some embodiments, a method for detecting glucose includes:
(i) applying a potential to a first working electrode of an analyte sensor, wherein the analyte sensor comprises:
   (a) a first working electrode;
   (b) a glutamate-responsive sensing layer disposed upon a surface of the first working electrode; and
   (c) a highly permeable membrane that overcoats at least a part of the glutamate-responsive sensing layer and that is permeable to glutamate; wherein the highly permeable membrane comprises a copolymer of poly(N-vinylimidazole) and poly(N-isopropylacrylamide);
wherein the analyte sensor shows a sensitivity of at least 100 nA/mM to glutamate;
(ii) obtaining a first signal at or above an oxidation-reduction potential of the first sensing layer, the first signal being proportional to a concentration of glutamate in a fluid contacting the first sensing layer; and
(iii) correlating the first signal to the concentration of glutamate in the fluid.

In some embodiments, the method of the present disclosure can further include detecting a second analyte by providing an analyte sensor that includes a second analyte-responsive sensing layer and/or exposing an analyte sensor that includes a second analyte-responsive sensing layer to a fluid comprising the first analyte and the second analyte. In some embodiments, the analyte sensor for use in a method for detecting a first analyte and a second analyte can further include a second working electrode; and a second analyte-responsive sensing layer disposed upon a surface of the second working electrode and responsive to a second analyte differing from the first analyte, where the second analyte-responsive sensing layer comprises a second polymer, at least one enzyme responsive to the second analyte covalently bonded to the second polymer and, optionally, a redox mediator covalently bonded to the second polymer; wherein a portion, e.g., second portion, of the mass transport limiting membrane overcoats the second analyte-responsive sensing layer. Alternatively, the second analyte-responsive active site can be covered by a second mass transport limiting membrane that is separate and/or different than a mass transport limiting membrane that overcoats the first analyte-responsive sensing layer. In some embodiments, at least one enzyme responsive to the second analyte comprises an enzyme system comprising multiple enzymes that are collectively responsive to the second analyte.

In some embodiments, the method further includes attaching an electronics unit to the skin of the patient, coupling conductive contacts of the electronics unit to contacts of the sensor, collecting data using the electronics unit regarding a level of analyte from signals generated by the sensor, and forwarding the collected data from electronics unit to a receiver unit, e.g., by RF. In some embodiments, the receiver unit is a mobile telephone. In some embodiments, the mobile telephone includes an application related to the monitored analyte. In some embodiments, analyte information is forwarded by RFID protocol, such as BLUETOOTH®, and the like.

In some embodiments, the analyte sensor can be positioned in a user for automatic analyte sensing, e.g., continuously or periodically. In some embodiments, the level of the analyte can be monitored over a time period ranging from seconds to minutes, hours, days, weeks or months. In some embodiments, the methods disclosed herein can be used to predict future levels of the analyte, based on the obtained information, such as but not limited to current analyte level at time zero, as well as the rate of change of the analyte concentration or amount.

### V. EXEMPLARY EMBODIMENTS

(1) In some non-limiting embodiments, the presently disclosed subject matter provides for analyte sensors comprising:
(i) first working electrode;
(ii) a first sensing layer disposed upon a surface of the first working electrode; and
(iii) a highly permeable membrane that overcoats at least a part of the first sensing layer and that is permeable to a first analyte; wherein the highly permeable membrane comprises a copolymer of poly(N-vinylimidazole) and poly(N-isopropylacrylamide);
wherein the analyte sensor shows a sensitivity of at least 100 nA/mM to the first analyte.

(2) The analyte sensor of (1), wherein the highly permeable membrane comprises a polymer exhibiting a lower critical solution temperature over a range from about 22° C. to about 42° C. in phosphate buffered saline.

(3) The analyte sensor of (1) or (2), wherein the highly permeable membrane comprises a copolymer with a poly(N-vinylimidazole) block having a number average molecular weight from about 2800 to about 4800 and a poly(N-isopropylacrylamide) block having a number average molecular weight from about 5600 to about 8000.

(4) The analyte sensor of (3), wherein the poly(N-vinylimidazole) block has a number average molecular weight from about 3300 to about 4200 and the poly(N-isopropylacrylamide) block has a number average molecular weight from about 6200 to about 7400.

(5) The analyte sensor of (3) or (4), wherein the poly(N-vinylacrylamide) block has a number average molecular weight of about 3800 and the poly(N-isopropyl acrylacmide) block has a number average molecular weight of about 6800.

(6) The analyte sensor of any one of (1)-(5), wherein the highly permeable membrane further comprises a crosslinking agent.

(7) The analyte sensor of (6), wherein the crosslinking agent is a polyethylene glycol diglycidylether (PEGDGE).

(8) The analyte sensor of (6) or (7), wherein the ratio (w/v) of crosslinking agent to the copolymer of poly(N-vinylimidazole) and poly(N-isopropylacrylamide) is from about 1:100 to about 1:1.

(9) The analyte sensor of any one of (6)-(8), wherein the ratio (w/v) of crosslinking agent to the copolymer of poly(N-vinylimidazole) and poly(N-isopropylacrylamide) is from about 1:40 to about 1:20.

(10) The analyte sensor of any one of (1)-(9), wherein the analyte sensor generates a signal that is substantially temperature independent over a range of temperatures.

(11) The analyte sensor of (10), wherein the range of temperatures is from about 25° C. to about 45° C.

(12) The analyte sensor of (11), wherein the analyte sensor generates a signal that varies by no more than 5% over the temperature range at a constant analyte concentration.

(13) The analyte sensor of any one of (1)-(12), wherein the analyte sensor shows a sensitivity of at least 150 nA/nM to the analyte.

(14) The analyte sensor of any one of (1)-(13), wherein the analyte sensor shows a sensitivity to an analyte that is greater than the sensitivity to the analyte shown by an otherwise identical sensor lacking a highly permeable membrane comprising a copolymer of poly(N-vinylimidazole) and poly(N-isopropylacrylamide).

(15) The analyte sensor of any one of (1)-(13), wherein the analyte sensor shows a sensitivity to an analyte that is at least 25% greater than the sensitivity to the analyte shown by an otherwise identical sensor lacking a highly permeable membrane comprising a copolymer of poly(N-vinylimidazole) and poly(N-isopropylacrylamide).

(16) The analyte sensor of any one of (1)-(15), wherein the analyte sensor generates a signal that varies by no more than 10% over the temperature range at a constant analyte concentration for at least 10 days.

(17) The analyte sensor of any one of (1)-(15), wherein the analyte sensor generates a signal that varies by no more than 10% over the temperature range at a constant analyte concentration for at least 15 days.

(18) The analyte sensor of any one of (1)-(17), wherein the first analyte is selected from the group consisting of glucose, glutamate, a ketone, an alcohol, lactate, and combinations thereof.

(19) The analyte sensor of any one of (1)-(18), wherein the first analyte is glucose.

(20) The analyte sensor of any one of (1)-(18), wherein the first analyte is

glutamate.

(21) The analyte sensor of any one of (1)-(20), wherein the first working electrode comprises carbon.

(22) The analyte sensor of any one of (1)-(21), wherein the first sensing layer further comprises a redox mediator.

(23) The analyte sensor of any one of (1)-(22), further comprising a reference electrode, a counter electrode, or both a reference electrode and a counter electrode.

(24) The analyte sensor of any one of (1)-(23), wherein the analyte sensor exhibits less than 20% delamination over a period of 12 days.

(25) The analyte sensor of any one of (1)-(24), wherein the analyte sensor exhibits less than 5% delamination over a period of 15 days.

(26) The analyte sensor of any one of (1)-(25), further comprising a second sensing layer disposed upon a surface of the first working electrode, the second sensing layer being responsive to a second analyte differing from the first analyte, wherein the first sensing layer comprises at least one enzyme responsive to the first analyte and the second sensing layer comprises at least one enzyme responsive to the second analyte.

(27) The analyte sensor of any one of (1)-(25), further comprising:
(iv) a second working electrode; and
(v) a second sensing layer disposed upon a surface of the second working electrode, the second sensing layer being responsive to a second analyte differing from the first analyte;
wherein the first sensing layer comprises at least one enzyme responsive to the first analyte and the second sensing layer comprises at least one enzyme responsive to the second analyte.

(28) The analyte sensor of (26) or (27), wherein a highly permeable membrane overcoats at least a part of the second sensing layer and is permeable to the second analyte; wherein the analyte sensor shows a sensitivity of at least 100 nA/mM to the second analyte.

(29) In some non-limiting embodiments, the presently disclosed subject matter provides for a method for monitoring a level of an analyte comprising:
(i) applying a potential to a first working electrode of an analyte sensor, wherein the analyte sensor comprises:
   (a) a first working electrode;
   (b) a first sensing layer disposed upon a surface of the first working electrode; and
   (c) a highly permeable membrane that overcoats at least a part of the first sensing layer and that is permeable to a first analyte, wherein the highly permeable membrane comprises a copolymer of poly(N-vinylimidazole) and poly(N-isopropylacrylamide);
wherein the analyte sensor shows a sensitivity of at least 100 nA/mM to the first analyte;
(ii) obtaining a first signal at or above an oxidation-reduction potential of the first sensing layer, the first signal being proportional to a concentration of the first analyte in a fluid contacting the first sensing layer; and
(iii) correlating the first signal to the concentration of the first analyte in the fluid.

(30) The method of (29), wherein the highly permeable membrane exhibits a lower critical solution temperature over a range from about 22° C. to about 42° C. in phosphate buffered saline.

### VI. ASPECTS OF THE DISCLOSURE

(31) An analyte sensor comprising:
(i) first working electrode;
(ii) a first sensing layer disposed upon a surface of the first working electrode; and
(iii) a highly permeable membrane that overcoats at least a part of the first sensing layer and that is permeable to a first analyte;
wherein the analyte sensor shows a sensitivity of at least 100 nA/mM to the first analyte.

(32) The analyte sensor of (31), wherein the highly permeable membrane comprises a polymer exhibiting a lower critical solution temperature over a range from about 22° C. to about 42° C. in phosphate buffered saline.

(33) The analyte sensor of (31) or (32), wherein the highly permeable membrane comprises a copolymer of poly(N-isopropyl acrylamide).

(34) The analyte sensor of (33), wherein the copolymer further comprises a polymer selected from the group consisting of poly(4-vinylpyridine), poly(N-vinylimidazole), poly(thiophene), poly(aniline), poly(pyrrole), and poly(acetylene).

(35) The analyte sensor of any one of (31)-(34), wherein the highly permeable membrane comprises a copolymer of poly(N-vinylimidazole) and poly(N-isopropyl acrylamide).

(36) The analyte sensor of any one of (31)-(35), wherein the highly permeable membrane comprises a copolymer with a poly(N-vinylimidazole) block having a number average molecular weight from about 2800 to about 4800 and a poly(N-isopropyl acrylamide) block having a number average molecular weight from about 5600 to about 8000.

(37) The analyte sensor of (36), wherein the poly(N-vinylimidazole) block has a number average molecular weight from about 3300 to about 4200 and the poly(N-isopropyl acrylamide) block has a number average molecular weight from about 6200 to about 7400.

(38) The analyte sensor of (36) or (37), wherein the poly(N-vinylacrylamide) block has a number average molecular weight of about 3800 and the poly(N-isopropyl acrylacmide) block has a number average molecular weight of about 6800.

(39) The analyte sensor of any one of (31)-(38), wherein the highly permeable membrane further comprises a crosslinking agent.

(40) The analyte sensor of any one of (31)-(39), wherein the crosslinking agent is a polyethylene glycol diglycidylether (PEGDGE).

(41) The analyte sensor of any one of (31)-(40), wherein the analyte sensor generates a signal that is substantially temperature independent over a range of temperatures.

(42) The analyte sensor of (41), wherein the range of temperatures is from about 25° C. to about 45° C.

(43) The analyte sensor of (42), wherein the analyte sensor generates a signal that varies by no more than 5% over the temperature range at a constant analyte concentration.

(44) The analyte sensor of any one of (41)-(43), wherein the analyte sensor shows a sensitivity of at least 150 nA/nM to the analyte.

(45) The analyte sensor of any one of (41)-(44), wherein the analyte sensor generates a signal that varies by no more than 10% over the temperature range at a constant analyte concentration for at least 10 days.

(46) The analyte sensor of any one of (41)-(44), wherein the analyte sensor generates a signal that varies by no more than 10% over the temperature range at a constant analyte concentration for at least 15 days.

(47) The analyte sensor of any one of (41)-(45), wherein the analyte is selected from the group consisting of glucose, glutamate, a ketone, an alcohol, lactate, and combinations thereof.

(48) The analyte sensor of any one of (31)-(47), wherein the analyte is glucose.

(49) The analyte sensor of any one of (31)-(47), wherein the analyte is glutamate.

(50) The analyte sensor of any one of (31)-(49), wherein the first working

electrode comprises carbon.

(51) The analyte sensor of any one of (31)-(50), wherein the first sensing layer further comprises a redox mediator.

(52) The analyte sensor of any one of (31)-(51), further comprising a reference electrode, a counter electrode, or both a reference electrode and a counter electrode.

(53) The analyte sensor of any one of (31)-(52), further comprising a second sensing layer disposed upon a surface of the first working electrode, the second sensing layer being responsive to a second analyte differing from the first analyte, wherein the second sensing layer comprises at least one enzyme responsive to the second analyte.

(54) The analyte sensor of any one of (31)-(53), further comprising:
a second working electrode; and
a second sensing layer disposed upon a surface of the second working electrode,
the second sensing layer being responsive to a second analyte differing from the first analyte;
wherein the second sensing layer comprises at least one enzyme responsive to the second analyte.

(55) The analyte sensor of (53) or (54), wherein a highly permeable membrane overcoats at least a part of the second sensing layer and is permeable to the second analyte; wherein the analyte sensor shows a sensitivity of at least 100 nA/mM to the second analyte.

(56) A method comprising:
applying a potential to a first working electrode of an analyte sensor, wherein the analyte sensor comprises:
a first working electrode;
a first sensing layer disposed upon a surface of the first working electrode; and
a highly permeable membrane that overcoats at least a part of the first sensing layer and that is permeable to a first analyte;
wherein the analyte sensor shows a sensitivity of at least 100 nA/mM to the analyte;
obtaining a first signal at or above an oxidation-reduction potential of the first sensing layer, the first signal being proportional to a concentration of a first analyte in a fluid contacting the first sensing layer; and
correlating the first signal to the concentration of the first analyte in the fluid.

(57) The method of (56), wherein the highly permeable membrane comprises a polymer exhibiting a lower critical solution temperature over a range from about 22° C. to about 42° C. in phosphate buffered saline.

### EXAMPLES

The presently disclosed subject matter will be better understood by reference to the following Examples, which are provided as exemplary of the presently disclosed subject matter, and not by way of limitation.

### Example 1: Stock Solution for Comparative Membranes

A 10Q5 stock solution was prepared by mixing 100 mg/mL of a 10Q5 polymer in 95:5 ratio (by volume) of ethanol: 10 mM N-2-hydroxyethylpiperazine-N′-2-ethanesulfonic acid (HEPES) buffer at a pH of 8.0. A Gly3 stock solution was prepared by mixing 12.5 mg/mL of triglycidyl glycerol (Gly3) in a 95:5 ratio (by volume) of ethanol: 10 mM HEPES buffer at a pH of 8.0. A PDMS stock solution was prepared by mixing 100 mg/mL of aminopropyl terminated polydimethylsiloxane (PDMS) in a 95:5 ratio (by volume) of ethanol: 10 mM HEPES buffer at a pH of 8.0.

### Example 2: Preparation of Glucose Sensor with Comparative Membrane

4 mL of the 10Q5 stock solution, 1 mL of the Gly3 stock solution, and 0.0165 mL of the PDMS stock solution were mixed together to provide a comparative membrane solution. The comparative membrane solution had a final measure (weight by volume) of 79.74 mg/mL of 10Q5, 2.49 mg/mL of Gly3, and 0.33 mg/mL of PDMS.

A comparative analyte sensor for glucose was washed in deionized water for 1 second and dried for 20 minutes before dipping. All dipping steps were conducted at 55% relative humidity and at a temperature of 21° C. After drying, the comparative glucose sensor was dipped 3 times with a 15 mm/sec (entry and exit) speed into the comparative membrane solution. The comparative glucose sensor was allowed to dry for 10 minutes after the 1st dip, 10 minutes after the 2nd dip, and 20 minutes after the 3rd dip. After dipping was complete, the glucose sensor was stored at 60% relative humidity and at a temperature of 25° C. for 24 hours to cure. After 24 hours, the comparative glucose sensor was transferred to a desiccated vial and aged at 56° C. for 24 hours. The resulting membrane had a thickness of 12 μm.

### Example 3: Stock Solutions for Highly Permeable Membranes for Glucose Sensors

A copolymer of poly(N-vinylimidazole) and poly(N-isopropylacrylamide) (PVINIPAA) stock solution was prepared by mixing 100 mg/mL of a copolymer of poly(N-vinylimidazole) (Mₙ=3800) and poly(N-isopropylacrylamide) (Mₙ=6800) in a 80:20 ratio (by volume) of ethanol: 10 mM HEPES buffer at a pH of 8.0. A polyethylene glycol diglycidyl ether (PEGDGE) 400 stock solution was prepared by mixing 100 mg/mL of PEGDGE 400 in a 80:20 ratio (by volume) of ethanol: 10 mM HEPES buffer at a pH of 8.

### Example 4: Preparation of Glucose Sensor with Highly Permeable Membrane A

4 mL of the PVINIPAA stock solution and 0.15 mL of the PEGDGE 400 stock solution were mixed together to provide a highly permeable membrane A solution. The highly permeable membrane A solution had a final measure (weight by volume) of 97.6 mg/mL of PVINIPAA and 2.4 mg/mL of PEGDGE 400.

An analyte sensor for glucose was washed in deionized water for 1 second and dried for 20 minutes before dipping. All dipping steps were conducted at 55% relative humidity and at a temperature of 21° C. After drying, the glucose sensor was dipped 3 times with a 10 mm/sec (entry and exit) speed into the highly permeable membrane A solution. The glucose sensor was allowed to dry for 10 minutes after the 1st dip, 10 minutes after the 2nd dip, and 20 minutes after the 3rd dip. After dipping was complete, the glucose sensor was stored at 60% relative humidity and at a temperature of 25° C. for 24 hours to cure. After 24 hours, the glucose sensor was transferred to a desiccated vial and aged at 56° C. for 24 hours. The resulting membrane had a thickness of 19 μm.

### Example 5: Preparation of Glucose Sensor with Highly Permeable Membrane B

4 mL of the PVINIPAA stock solution and 0.1 mL of the PEGDGE 400 stock solution were mixed together to provide a highly permeable membrane B solution. The highly permeable membrane B solution had a final measure (weight by volume) of 96.4 mg/mL of PVINIPAA and 3.6 mg/mL of PEGDGE 400.

An analyte sensor for glucose was washed in deionized water for 1 second and dried for 20 minutes before dipping. All dipping steps were conducted at 55% relative humidity and at a temperature of 21° C. After drying, the glucose sensor was dipped 3 times with a 10 mm/sec (entry and exit) speed into the highly permeable membrane B solution. The glucose sensor was allowed to dry for 10 minutes after the 1st dip, 10 minutes after the 2nd dip, and 20 minutes after the 3rd dip. After dipping was complete, the glucose sensor was stored at 60% relative humidity and at a temperature of 25° C. for 24 hours to cure. After 24 hours, the glucose sensor was transferred to a desiccated vial and aged at 56° C. for 24 hours. The resulting membrane had a thickness of 18 μm.

### Example 6: Beaker Stability of the Glucose Sensors

The comparative glucose sensor and the sensors prepared with highly permeable membranes were made with the same sensing layer composition that was deposited multiple times on a substrate, which was then cut to form a single sensor.

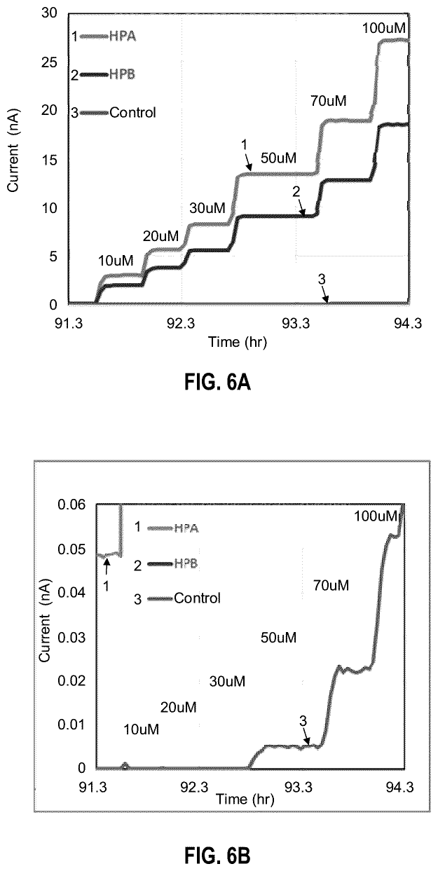

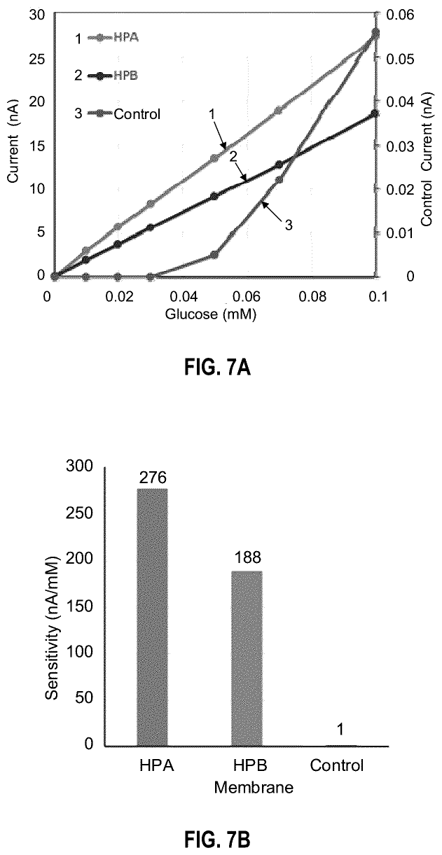

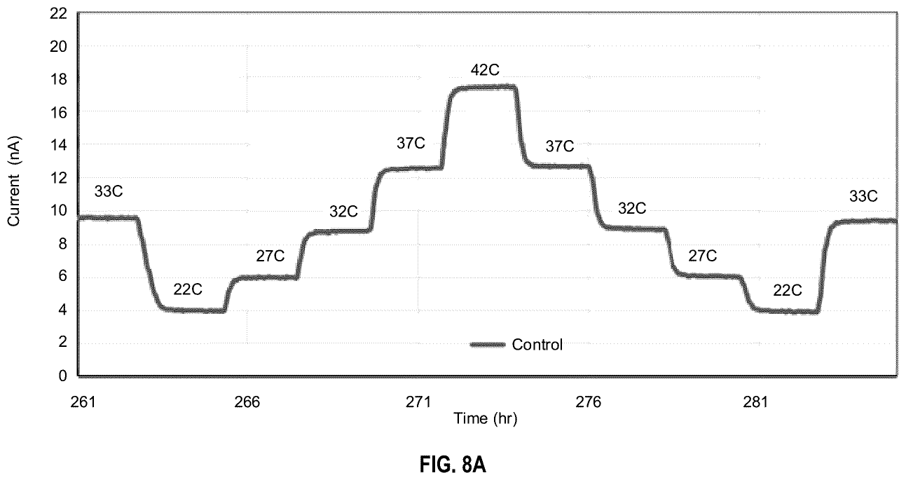

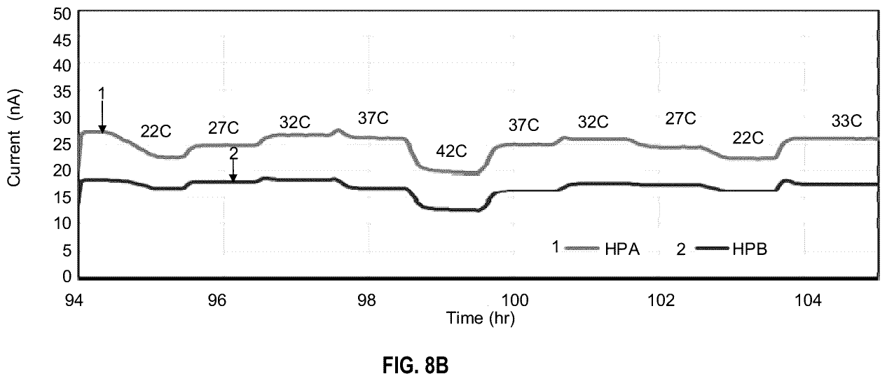

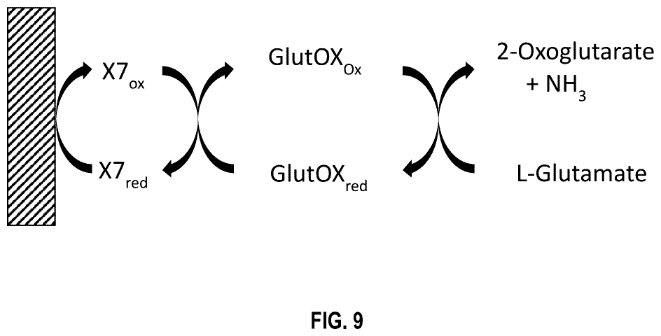

The beaker stability (long-term stability) of the glucose sensor with highly permeable membrane A, the glucose sensor with highly permeable membrane B, and the comparative glucose sensor was evaluated over 3 hours. Into a beaker containing the glucose sensors in a solution of phosphate-buffered saline (PBS) with a pH of 7.4 at 33° C., was added 10 μm of glucose at 20 minutes, an additional 10 μm of glucose at 40 minutes, an additional 10 μm of glucose at 1 hour, an additional 20 μm of glucose at 1.3 hours, an additional 20 μm of glucose at 2.2 hours, and an additional 30 μm of glucose at 2.6 hours, with a final measurement taken at 3 hours. The results, shown in FIGS. 6A and 6B, demonstrate that the glucose sensors prepared with a highly permeable membrane were much more sensitive to an increase in glucose concentration than the comparative glucose sensor that did not comprise a highly permeable membrane. And, as shown in FIGS. 7A and 7B, the glucose sensors with a highly permeable membrane were 276 (for highly permeable membrane A) and 188 (for highly permeable membrane B) times more sensitive to glucose than the comparative glucose sensor. Thus, the sensitivity of the glucose sensors with a highly permeable membrane is significantly better than that of the control glucose sensor.

### Example 7: Preparation of Glutamate Sensor with Comparative Membrane

4 mL of the 10Q5 stock solution, 1 mL of the Gly3 stock solution, and 0.0165 mL of the PDMS stock solution were mixed together to provide a comparative membrane solution. The comparative membrane solution had a final measure (weight by volume) of 79.74 mg/mL of 10Q5, 2.49 mg/mL of Gly3, and 0.33 mg/mL of PDMS.

A comparative analyte sensor for glutamate was washed in deionized water for 1 second and dried for 20 minutes before dipping. All dipping steps were conducted at 55% relative humidity and at a temperature of 21° C. After drying, the comparative glutamate sensor was dipped 3 times with a 15 mm/sec (entry and exit) speed into the comparative membrane solution. The comparative glutamate sensor was allowed to dry for 10 minutes after the 1st dip, 10 minutes after the 2nd dip, and 20 minutes after the 3rd dip. After dipping was complete, the glutamate sensor was stored at 60% relative humidity and at a temperature of 25° C. for 24 hours to cure. After 24 hours, the comparative glutamate sensor was transferred to a desiccated vial and aged at 56° C. for 24 hours. The resulting membrane had a thickness of 12 μm.

### Example 8: Stock Solutions for Highly Permeable Membranes for Glutamate Sensors

A PVINIPAA stock solution was prepared by mixing 100 mg/mL of PVINIPAA in a 80:20 ratio (by volume) of ethanol: 10 mM HEPES buffer at a pH of 8.0. A stock solution of PEGDGE 1000 was prepared by mixing 200 mg/mL of PEGDGE 1000 in a 80:20 ratio (by volume) of ethanol: 10 mM HEPES buffer at a pH of 8.

### Example 9: Preparation of Glutamate Sensor with Highly Permeable Membrane C

4 mL of the PVINIPAA stock solution and 0.2 mL of the PEGDGE 1000 stock solution were mixed together to provide a highly permeable membrane C solution. The highly permeable membrane C solution had a final measure (weight by volume) of 95.24 mg/mL of PVINIPAA and 9.52 mg/mL of PEGDGE 1000.

An analyte sensor for glutamate was washed in deionized water for 1 second and dried for 20 minutes before dipping. All dipping steps were conducted at 55% relative humidity and at a temperature of 21° C. After drying, the analyte sensor was dipped 3 times with a 15 mm/sec (entry and exit) speed into the highly permeable membrane C solution. The analyte sensor was allowed to dry for 10 minutes after the 1st dip, 10 minutes after the 2nd dip, and 20 minutes after the 3rd dip. After dipping was complete, the analyte sensor was stored at 60% relative humidity and at a temperature of 25° C. for 24 hours to cure. After 24 hours, the analyte sensor was transferred to a desiccated vial and aged at 56° C. for 24 hours. The resulting membrane had a thickness of 29 μm.

### Example 10: Beaker Stability of the Glutamate Sensors

The comparative glutamate sensor and the glutamate sensor prepared with highly permeable membrane C were made with the same sensing layer composition that was deposited multiple times on a substrate, which was then cut to form a single sensor.

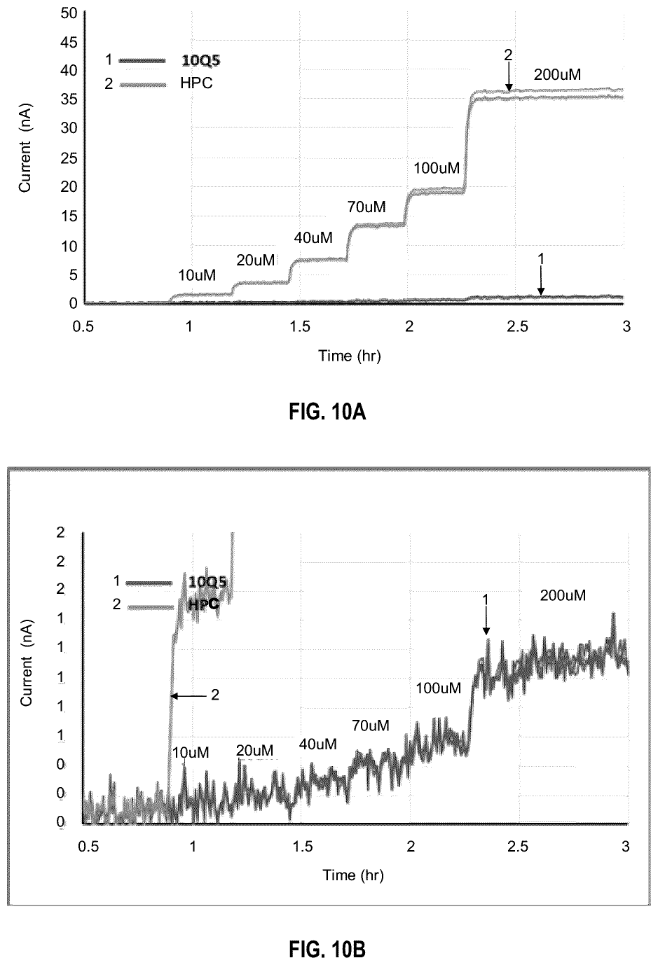

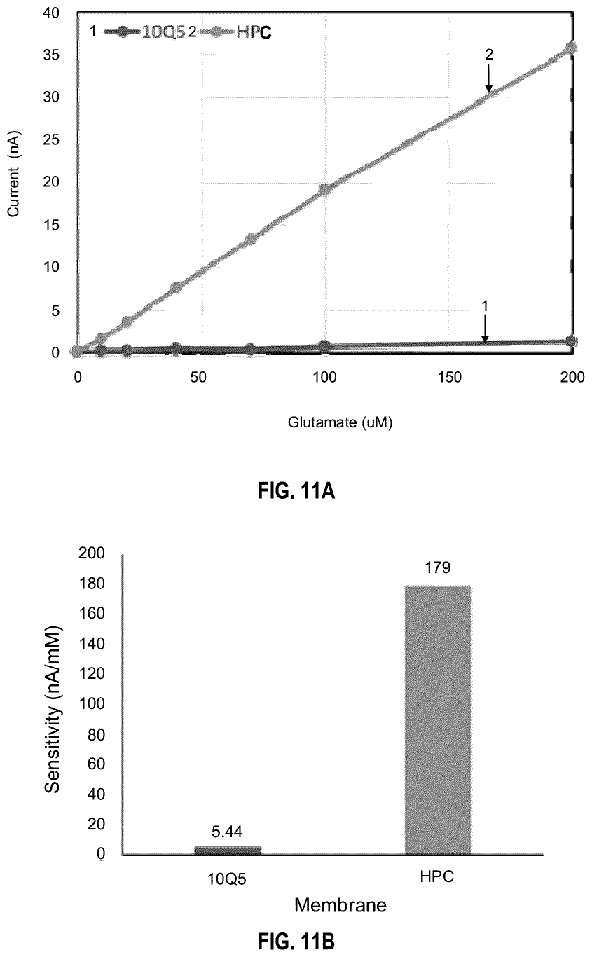

The beaker stability (long-term stability) of the glutamate sensor with highly permeable membrane C and the glutamate sensor with a comparative membrane was evaluated over 2.5 hours. Into a beaker containing the glutamate sensors in a solution of phosphatebuffered saline (PBS) with a pH of 7.4 at 33° C., was added 10 μm of glutamate at 20 minutes, an additional 10 μm of glutamate at 40 minutes, an additional 20 μm of glutamate at 1.0 hour, an additional 30 μm of glutamate at 1.2 hours, an additional 30 μm of glutamate at 1.5 hours, and an additional 100 μm of glutamate at 1.8 hours, with a final measurement taken at 2.5 hours. The results, shown in FIGS. 10A and 10B, demonstrate that the glutamate sensor with highly permeable membrane C was much more sensitive to an increase in glutamate concentration than the comparative glutamate sensor. And, as shown in FIGS. 11A and 11B, the glutamate sensor with highly permeable membrane C exhibited a sensitivity of 179 nA/mM versus the 5.44 nA/mM exhibited by the comparative glutamate sensor. Thus, the glutamate sensor with highly permeable membrane C is significantly more sensitive than the comparative glutamate sensor.

### Example 11: Long-term Stability of Glutamate Sensor with Highly Permeable Membrane C

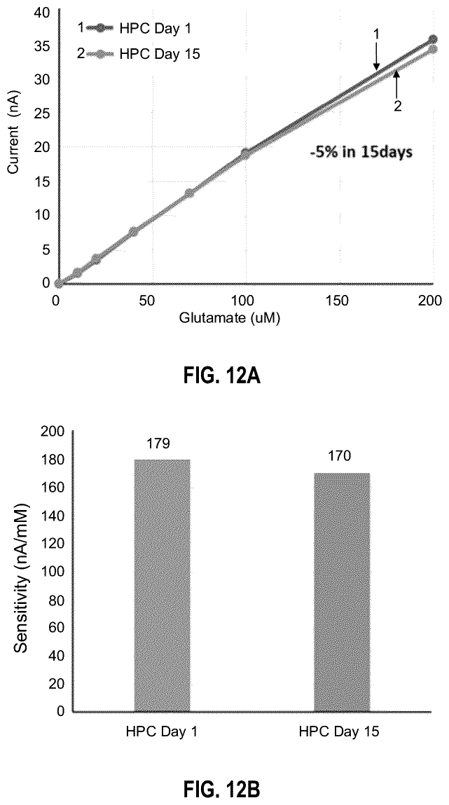

A long-term stability study on the glutamate sensor with highly permeable membrane C showed a 5% decrease in current (signal drop) after 15 days in FIG. 12A with a sensitivity of 179 nA/mM on Day 1 of testing and a sensitivity of 170 nA/mM on Day 15 of testing (FIG. 12B).

### Example 12: Preparation and Beaker Testing of Glucose Sensors

Glucose sensors having a working electrode that includes glucose oxidase (GOX) and flavin adenine dinucleotide (FAD) in the sensing layer were prepared. The sensing layer was coated with one of the membrane compositions described in TABLE 1.

A glucose sensor coated with a membrane comprising poly(4-vinylpyridine-co-N-isopropylacrylamide) (PVP-co-NIPAA) crosslinked with polyethylene glycol diglycidyl ether (PEGDGE) 1000 in the (w/v) ratio shown in TABLE 1 was prepared. The membrane comprised 60% N-isopropylacrylamide (NIPAA) by monomer ratio (molar ratio). The membrane thickness was about 21.2 μm.

A glucose sensor coated with a membrane comprising poly(4-vinylimidazole-co-N-isopropylacrylamide) (PVI-co-NIPAA) crosslinked with polyethylene glycol diglycidyl ether (PEGDGE) 400 in the (w/v) ratio shown in TABLE 1 was prepared. The membrane comprised 60% NIPAA by monomer ratio (molar ratio). The membrane thickness was about 19.0 μm.

TABLE 1
Membrane Compositions for Glucose Sensors

Final (mg/mL)

| Reagent | PVP co NIPAA | PVI co NIPAA |
|---|---|---|
| PVP-co-NIPAA (60%) | 92 | 0 |
| PVI-co-NIPAA (60%) | 0 | 96 |
| PEGDGE 1000 | 16 | 0 |
| PEGDGE 400 | 0 | 4 |
| % Crosslinker | 18% | 4% |
| Membrane Thickness (μm) | 21.2 | 19.0 |

The glucose sensors were tested in a solution of 0.1 M phosphate buffer (PBS) including 0.1 M NaCl to arrive at a pH=7.4 with 10 mM glucose at a temperature of 33° C. The temperature was controlled by a circulated water system with a digital temperature controller.

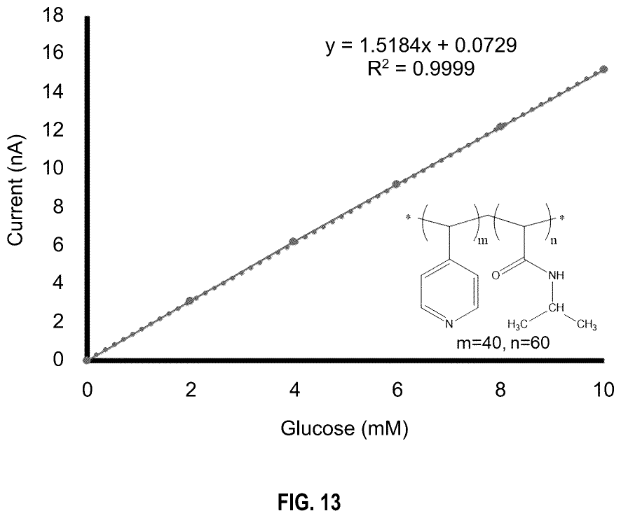

FIG. 13 provides a sensitivity curve for a glucose sensor comprising a working electrode having glucose oxidase (GOX) and flavin adenine dinucleotide (FAD) in the sensing layer and coated with a poly(4-vinylpyridine-co-N-isopropylacrylamide) membrane. As shown in FIG. 13, a concentration of glucose of about 10 mM resulted in current of about 16 nA which corresponds to a sensitivity of 1.5 nA/mM for the glucose sensor.

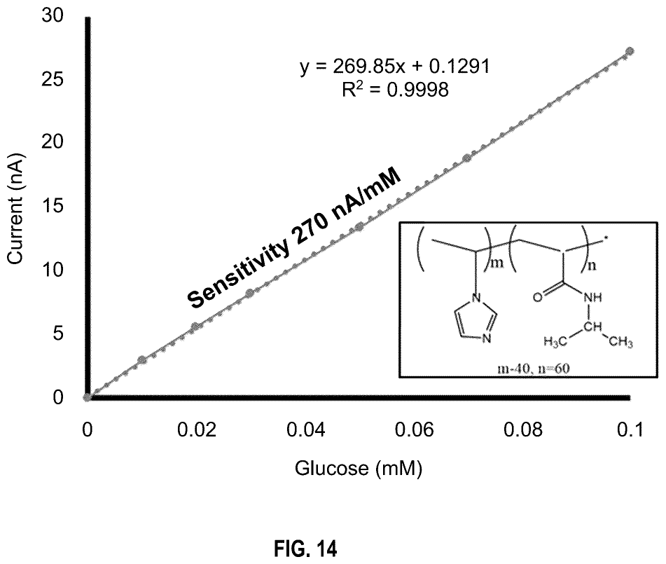

FIG. 14 provides a sensitivity curve for a glucose sensor comprising a working electrode having glucose oxidase (GOX) and flavin adenine dinucleotide (FAD) in the sensing layer and coated with poly(4-vinylimidazole-co-N-isopropylacrylamide) membrane. As shown in FIG. 14, a concentration of glucose of about 0.1 mM resulted in a current of about 17 nA which corresponds to a sensitivity of 270 nA/mM for the glucose sensor. Therefore, the glucose sensor comprising a poly(4-vinylimidazole-co-N-isopropylacrylamide) membrane is much more sensitive to glucose.

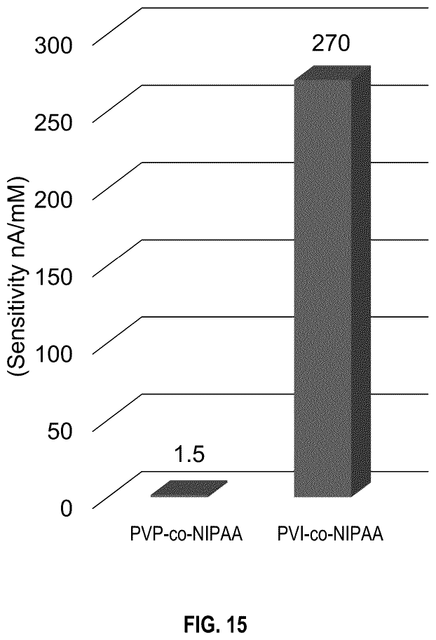

The difference in sensitivity is also shown in FIG. 15, in which the glucose sensor having a poly(4-vinylimidazole-co-N-isopropylacrylamide) membrane coated onto a working electrode exhibited a sensitivity 180 times that of the glucose sensor having a poly(4-vinylpyridine-co-N-isopropylacrylamide) membrane coated onto a working electrode.

### Example 13: Beaker Testing Results with Other Polymer Membranes

Additional analyte sensors coated with: (1) a poly(4-vinylpyridine-co-styrene sulfuric acid) (PVP-co-PSS) comprising 50% styrene sulfuric acid by monomer ratio (molar ratio); (2) poly(acrylic acid-co-N-isopropylacrylamide) (PAA-co-NIPAA) comprising 50% NIPAA by monomer ratio (molar ratio); and (3) polyurethane, were prepared and the beaker test results are provided in TABLE 2.

TABLE 2
Beaker Test Results for Additional Polymer Membranes

| Polymer | Analyte | Sensitivity | | |
|---|---|---|---|---|
| Membrane | Tested | (nA/mM) | | Result |
| PVP-co-PSS | Glutamate | ~100 | nA/mM | Delamination after |
| (50%) | | | | soaking in PBS |
| PAA-co-NIPAA | Glucose | ~50 | nA/mM | Delamination after |
| (50%) | | | | soaking in PBS; |
| | | | | Membrane was |
| | | | | brittle |
| Polyurethane | Glutamate | ~100-150 | nA/mM | Delamination after |
| | | | | soaking in PBS |

The analyte sensors were tested in a solution of 0.1 M phosphate buffer (PBS) including 0.1 M NaCl to arrive at a pH=7.4 with 10 mM analyte at a temperature of 33° C. The temperature was controlled by a circulated water system with a digital temperature controller.

Although analyte sensors prepared with a PVP-co-PSS membrane and with a polyurethane membrane were found to provide high sensitivity to glutamate, soaking these analyte sensors in a buffer solution resulted in delamination of the membrane. Alternatively, analyte sensors comprising PVI-co-NIPAA did not shown delamination of the membrane after soaking in PBS.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

1. An in vivo analyte sensor for measuring analyte levels in a bodily fluid of a user comprising:
a first portion coupled with a sensor control unit, wherein the first portion is positionable above a surface of a skin,
a second portion positionable below the surface of the skin, wherein the second portion is in contact with bodily fluid and configured to measure signals indicative of analyte levels in the bodily fluid, wherein the second portion comprises electrodes connected to contact portions positioned on the first portion;
wherein the sensor control unit comprises
a processor configured to determine data indicative of analyte levels and to transmit the data indicative of analyte levels to a receiver unit according to a Bluetooth communication protocol via a transmitter coupled to the processor, and
a power supply for operating the sensor control unit;
wherein the electrodes of the second portion include
(i) a first working electrode;
(ii) a first sensing area disposed upon a surface of the first working electrode; and
(iii) a highly permeable membrane that overcoats at least a part of the first sensing area and that is permeable to a first analyte, wherein the highly permeable membrane comprises a copolymer of poly(N-vinylimidazole) and poly(N-isopropylacrylamide); and
a counter electrode;
wherein the sensor control unit is configured to process factory-determined calibrated measurements that are input or stored in the sensor control unit, such that the sensor is thereby factory calibrated; and
wherein the analyte sensor shows a sensitivity of at least 100 nA/mM to the first analyte.

2. The analyte sensor of claim 1, wherein the highly permeable membrane exhibits a lower critical solution temperature over a range from about 22° C. to about 42° C. in phosphate buffered saline.

3. The analyte sensor of claim 1, wherein the highly permeable membrane comprises a copolymer with a poly(N-vinylimidazole) block having a number average molecular weight from about 2800 to about 4800 and a poly(N-isopropylacrylamide) block having a number average molecular weight from about 5600 to about 8000.

4. The analyte sensor of claim 3, wherein the poly(N-vinylimidazole) block has a number average molecular weight from about 3300 to about 4200 and the poly(N-isopropylacrylamide) block has a number average molecular weight from about 6200 to about 7400.

5. The analyte sensor of claim 3, wherein the poly(N-vinylacrylamide) block has a number average molecular weight of about 3800 and the poly(N-isopropyl acrylacmide) block has a number average molecular weight of about 6800.

6. The analyte sensor of claim 1, wherein the highly permeable membrane further comprises a crosslinking agent.

7. The analyte sensor of claim 6, wherein the crosslinking agent is a polyethylene glycol diglycidylether (PEGDGE).

8. The analyte sensor of claim 6, wherein the crosslinking agent and the copolymer of poly(N-vinylimidazole) and poly(N-isopropylacrylamide) are present in a ratio (w/v) from about 1:100 to about 1:1.

9. The analyte sensor of claim 6, wherein the crosslinking agent and the copolymer of poly(N-vinylimidazole) and poly(N-isopropylacrylamide) are present in a ratio (w/v) from about 1:40 to about 1:20.

10. The analyte sensor of claim 1, wherein the analyte sensor generates a signal that is substantially temperature independent over a range of temperatures.

11. The analyte sensor of claim 10, wherein the range of temperatures is from about 25° C. to about 45° C.

12. The analyte sensor of claim 11, wherein the analyte sensor generates a signal that varies by no more than 5% over the temperature range at a constant analyte concentration.

13. The analyte sensor of claim 1, wherein the analyte sensor shows a sensitivity of at least 150 nA/nM to the first analyte.

14. The analyte sensor of claim 1, wherein the analyte sensor shows a sensitivity to an analyte that is greater than the sensitivity to the analyte shown by an otherwise identical sensor lacking a highly permeable membrane comprising a copolymer of poly(N-vinylimidazole) and poly(N-isopropylacrylamide).

15. The analyte sensor of claim 1, wherein the analyte sensor shows a sensitivity to an analyte that is at least 25% greater than the sensitivity to the analyte shown by an otherwise identical sensor lacking a highly permeable membrane comprising a copolymer of poly(N-vinylimidazole) and poly(N-isopropylacrylamide).

16. The analyte sensor of claim 1, wherein the analyte sensor generates a signal that varies by no more than 10% over the temperature range at a constant analyte concentration for at least 10 days.

17. The analyte sensor of claim 1 wherein the analyte sensor generates a signal that varies by no more than 10% over the temperature range at a constant analyte concentration for at least 15 days.

18. The analyte sensor of claim 1, wherein the first analyte is selected from the group consisting of glucose, glutamate, a ketone, an alcohol, lactate, and combinations thereof.

19. The analyte sensor of claim 1, wherein the first analyte is glucose.

20. The analyte sensor of claim 1, wherein the first analyte is glutamate.

21. The analyte sensor of claim 1, wherein the first working electrode comprises carbon.

22. The analyte sensor of claim 1, wherein the first sensing area further comprises a redox mediator.

23. The analyte sensor of claim 1, further comprising a reference electrode, a counter electrode, or both a reference electrode and a counter electrode.

24. The analyte sensor of claim 1, wherein the analyte sensor exhibits less than 20% delamination over a period of 12 days.

25. The analyte sensor of claim 1, wherein the analyte sensor exhibits less than 5% delamination over a period of 15 days.

26. (canceled)

27. The analyte sensor of claim 1, further comprising:
(iv) a second working electrode; and
(v) a second sensing area disposed upon a surface of the second working electrode, the second sensing area being responsive to a second analyte differing from the first analyte;
wherein the first sensing area comprises at least one enzyme responsive to the first analyte and the second sensing area comprises at least one enzyme responsive to the second analyte.

28. The analyte sensor of claim 27, wherein a highly permeable membrane overcoats at least a part of the second sensing area and is permeable to the second analyte; and wherein the analyte sensor shows a sensitivity of at least 100 nA/mM to the second analyte.

29. A method for monitoring an analyte level in a bodily fluid of a user of an in vivo analyte sensor comprising:
(i) applying a potential to a first working electrode of an analyte sensor, wherein the analyte sensor comprises:
a first portion coupled with a sensor control unit, wherein the first portion is positionable above a surface of a skin,
a second portion positionable below the surface of the skin, wherein the second portion is in contact with bodily fluid and configured to measure signals indicative of analyte levels in the bodily fluid, wherein the second portion comprises electrodes connected to contact portions positioned on the first portion;
wherein the sensor control unit comprises
a processor configured to determine data indicative of analyte levels and to transmit the data indicative of analyte levels to a receiver unit according to a Bluetooth communication protocol via a transmitter coupled to the processor; and
a power supply for operating the sensor control unit;
wherein the electrodes of the second portion include
(a) a first working electrode;
(b) a first sensing area disposed upon a surface of the first working electrode; and
(c) a highly permeable membrane that overcoats at least a part of the first sensing area and that is permeable to a first analyte, wherein the highly permeable membrane comprises a copolymer of poly(N-vinylimidazole) and poly(N-isopropylacrylamide);
wherein the analyte sensor shows a sensitivity of at least 100 nA/mM to the analyte; and
a counter electrode;
wherein the sensor control unit is configured to process factory-determined calibrated measurements that are input or stored in the sensor control unit, such that the sensor is thereby factory calibrated;
(ii) obtaining a first signal at or above an oxidation-reduction potential of the first sensing area, the first signal being proportional to a concentration of the first analyte in the bodily fluid contacting the first sensing area; and
(iii) correlating the first signal to the concentration of the first analyte in the bodily fluid.

30. The method of claim 29, wherein the highly permeable membrane exhibits a lower critical solution temperature over a range from about 22° C. to about 42° C. in phosphate buffered saline.
